(19) United States
(12) Patent Application Publication
(10) Pub. No.: US 2004/0209263 A1
(43) Pub. Date: Oct. 21, 2004

(54) Selection of catalytic nucleic acids targeted to infectious agents

(76) Inventors: Gary A. Clawson, Bethesda, MD (US); Wei-Hua Pan, Hummelstown, PA (US)

Correspondence Address: Fish & Richardson, 225 Franklin Street, Boston, MA, 02110-2804, US

(21) Appl. No.: 10/433,542

(22) PCT Filed: Dec. 7, 2001
(86) PCT No.: PCT/US01/46178

Related U.S. Application Data
(60) Provisional application No. 60/251,810, filed on Dec. 7, 2000.

(51) Int. Cl. C12Q 1/68

(52) U.S. Cl. 435/6

(57) ABSTRACT

The invention provides improved library selection procedures for nucleic acids which allow the rapid determination of accessible target sites throughout relatively long target RNAs. This invention provides an improved method of screening a library of nucleic acids to identify cleavage sites of a target RNA. The steps of the screening comprise generating the library of nucleic acids, wherein each nucleic acid comprises a catalytic core flanked by random nucleotides; adding said target RNA to the library of nucleic acids; and isolating nucleic acids that cleave said target RNA. The nucleic acids selected by the methods described herein are also provided in the invention.

### FIELD OF THE INVENTION

The invention provides improved library selection procedures for nucleic acids which allow the rapid determination of accessible target sites throughout relatively long target RNAs.

The invention describes the selection of nucleic acids targeted to virtually any RNA including, but not limited to, eukaryotic and prokaryotic RNA, RNA from plants, mammals, fungi and various pathogenic organisms such as bacteria and viruses. Pathogenic viruses include, but are not limited to hepatitis B virus (HBV), hepatitis C virus (HCV), human immunodeficiency virus (HIV), and human papillomavirus (HPV). The selected nucleic acids comprise antisense oligonucleotides reverse complementary to identified cleavage sites, especially ribozymes with catalytic activity against RNAs.

### BACKGROUND OF THE INVENTION

All references and patents cited herein are hereby incorporated by reference in their entireties. It must be noted that as used in this specification and the appended claims, the singular forms “a”, “an” and “the” include plural referents unless the context clearly dictates otherwise. Thus, for example, reference to “a method of identifying one or more cleavage sites in a target RNA” includes one or more methods or steps of the type described herein.

A major limitation to the effectiveness of ribozymes is definition of accessible sites in targeted RNAs. Although library selection procedures have been developed, they have generally required labor-intensive cloning and sequencing to identify potential ribozyme cleavage sites. The present invention is directed to a selection technology that utilizes a randomized, active hammerhead ribozyme (Rz) library. After 1 or 2 rounds of binding under inactive conditions, the selected, active Rz library is incubated with target RNA, and the sites of cleavage are identified on sequencing gels. Rz targeted to sites identified with this procedure are generally more active than those identified with previously described oligonucleotide library selection procedures. The ribozymes are also more active in cell culture than ribozymes identified using other techniques.

Catalytic nucleic acids may be operationally divided into two components, a conserved stem-loop structure forming the catalytic core and flanking sequences which are reverse complementary to sequences surrounding the target site in a given RNA transcript. For example, Rz-mediated cleavage occurs just 3′ to a targeted nucleotide triplet, which can be NUH (N can be any nucleotide, but is often G, with H being A, C, or U). Flanking sequences confer specificity and generally constitute 14-16 nucleotides in total, extending on both sides of the target site selected; this allows sufficient specificity for the cleavage reaction while allowing ready dissociation from the target, which is typically the rate limiting step in the catalytic cycle (Goodchild & Kohli, 1991, Arch Biochem Biophys 284:386-391; Hendry & McCall, 1996, Nucl Acids Res 24:2679-2687; and Parker et al., 1992, Ribozymes: principles and designs for their use as antisense and therapeutic agents. In Gene Regulation: Biology of Antisense RNA and DNA. New York: Raven Press, ed. R. Erickson, J. Lzant pp. 55-70). Since the consensus triplet sequence is not very restrictive, virtually any RNA including any viral RNA of interest is likely to possess numerous potential target sites for Rz cleavage (Benedict et al., 1998, Carcinogenesis 19:1223-1230; Crone et al., 1999, Hepatology 29:1114-1123; Eldadah et al., 2000, J Neurosci 20:179-186; Folini et al., 2000, J Invest Dermatol 114:259-267; Macejak et al., 2000, Hepatology 31:769-776; Passman et al., 2000, Biochem Biophys Res Commun 268:728-733; Perlman et al., 2000, Cardiovasc Res 45:570-578; Ren et al., 1999, Gene Ther Mol Biol 3:257-269; Salmi et al., 2000, Eur J Pharmacol 388:R1-R2; and Suzuki et al., 2000, Gene Ther 7:241-248). To date, most investigators have selected target sites through a computer-aided process that searches for regions predicted to be single-stranded regions that contain a suitable nucleotide triplet. Unfortunately, it is frequently observed that only a small fraction of ribozymes engineered in this manner give rise to significant reductions in target RNA levels within cells.

Experimental approaches for identification of cleavable sites offer clear advantages. Lieber & Strauss constructed a library of hammerhead Rz that were targeted to a preselected triplet, and contained randomized sequences in the annealing arms, which allowed the screening of accessible sites in the target-RNA molecule (Lieber & Strauss, 1995, Mol Cell Biol 15:540-551). In this case, the selected Rz cleaved an in vitro transcript efficiently and inhibited gene expression strongly in cell culture; one selected Rz was successfully used for inhibition of growth-hormone expression in mice (Lieber & Kay, 1996, J Virol 70:3153-3158). In a different approach, Birikh et al. (Bikrikh et al., 1997, Eur J Biochem 245:1-16) used a completely randomized oligonucleotide (dN10) in conjunction with RNase H to map sites that are accessible for oligonucleotide binding in an RNA transcript: the best Rz generated in this fashion was 150-fold more active than the most efficient Rz designed on the basis of the mFold program which is a well-known computer sequence characterization program (Zuker & Stiegler, 1981, Nucleic Acids Res 9:133-148).

As a remarkably powerful tool, the systematic evolution of ligands by exponential enrichment, generically known as “SELEX”, (Ellington & Szostak, 1990, Nature 346:818-822 and Tuerk & Gold, 1990, Science 249:505-510) has been used to isolate oligonucleotide sequences, so-called aptamers, with the capacity to recognize virtually any class of target molecules with high affinity and specificity, such as organic dyes, amino acids, biological cofactors, antibiotics, peptides and proteins, or even whole viruses and protozoan organisms (Bell et al., 1998, J Biol Chem 273:14309-14314; Eaton, 1997, Curr Opin Chem Biol 1:10-16; Gal et al., 1998, Eur J Biochem 252:553-562; Homann & Goringer, 1999, Nucleic Acids Res 27:2006-2014; Kraus et al., 1998, J. Immunol. 160:5209-5212; Osborne & Ellington, 1997, Chem Rev 97:349-370; Pan et al., 1995, Proc Natl Acad Sci USA 92:11509-11513; Wang et al., 2000, RNA 6:571-583; and Yang et al., 1998, Proc Natl Acad Sci USA 95:5462-5467).

The effectiveness of catalytic nucleic acids is greatly influenced by the accessibility of selected targets sites in targeted RNAs. Procedures, which may incorporate the methods described above, have been designed which are referred to as “library selection” procedures, where a random pool of catalytic nucleic acids is mixed with the target RNA, and major cut products are then identified on sequencing gels. However, there are limitations to these procedures, particularly with regard to identifying good target sites away from the 5′ end of the targets. Furthermore, no such descriptions have appeared for DNAzymes (Dz), and it is not entirely clear that highly functional sites for Rz will necessarily be highly functional sites for Dz.

Accordingly, there is a need in the art for improved screening techniques for the identification of accessible sites in target RNAs for recognition by nucleic acids. Library screening procedures which utilize antisense oligonucleotides, Rz, and/or Dz, and overcome limitations in previous descriptions with regard to identification of target sites in more 3′ regions of target molecules, are described herein.

### SUMMARY OF THE INVENTION

The present invention is directed to methods of identifying one or more cleavage sites in a target RNA which are accessible to a ribozyme, said methods comprising:

(a) generating a library of RNAs, wherein each RNA in said library comprises a catalytically active hammerhead ribozyme core, wherein said ribozyme core is flanked on each side by random nucleotide regions, wherein said random nucleotide regions are flanked on each side by fixed sequences which allow amplification and a sequence which allows transcription of said RNA;

(b) contacting said target RNA with said library of RNAs under conditions in which said ribozyme core is not catalytically active;

(c) separating RNAs that bind to said target RNA from RNAs that do not bind;

(d) generating an enriched library of RNAs comprising RNAs bound in step (c);

(e) repeating steps (a) through (d) at least one additional time with a reduced ratio of said target RNA to said library of RNAs;

(f) generating 5′ or 3′ end-labeled target RNA;

(g) contacting said 5′ or 3′ end-labeled target RNA of step (f) with an enriched library of RNAs of step (e) under conditions in which said ribozyme core of said library of RNAs is catalytically active such that said target RNA is cleaved to produce cleavage products;

(h) separating said cleavage products from step (g) and determining the sequence or sequences at which cleavage of said end-labeled target RNA occurred as a result of incubation of said end-labeled target RNA with said library of RNAs.

In an embodiment, the methods of the invention are such that said target RNA of step (f) above is 3′ end-labeled target RNA.

In a preferred embodiment, the methods of the invention are such that said 3′ end-labeled target RNA is of uniform length and is produced by a method comprising:

(a) constructing a target RNA containing a 3′ cis-acting catalytic ribozyme having a 3′ flanking sequence that is reverse complementary to the 3′ end of said target RNA;

(b) cleaving said target RNA at the 3′ end with said 3′ cis-acting catalytic ribozyme; and

(c) labeling said target RNA at the 3′ end produced in step (b);

wherein said target RNA labeled in step (c) is 3′ end-labeled target RNA of uniform length.

In an embodiment of the present invention, the random nucleotide regions in step (a) above are about six to about twelve nucleotides in length. In a further embodiment, the random nucleotide regions in step (a) above are about seven to about eleven nucleotides in length. In a further embodiment, the random nucleotide regions in step (a) above are about eight to about ten nucleotides in length. In a still further embodiment, the random nucleotide regions in step (a) above are about nine nucleotides in length.

In an embodiment of the present invention, the sequence which allows transcription of the RNA is an Sp6 RNA promoter.

In an embodiment of the present invention, the condition in which the ribozyme core is not catalytically active of step (b) above is in the absence of Mg²⁺.

In an embodiment of the present invention, the separating of step (c) above is performed using electrophoretic chromatography or column chromatography.

In an embodiment of the present invention, the enriched library of RNAs of step (d) above is generated by PCR amplification of the RNA that binds in step (c).

In an embodiment of the present invention, repeating steps (a) through (d) above is done at least two additional times.

In an embodiment of the present invention, repeating steps (a) through (d) above is done at least three additional times.

In an embodiment of the present invention, repeating steps (a) through (d) above is done at least four additional times.

In an embodiment of the present invention, the conditions in which said ribozyme core of said library of RNAs is catalytically active of step (g) above is in the presence of Mg²⁺.

In an embodiment of the present invention, the separating of said cleavage products and determining the sequence or sequences at which cleavage of said target RNA occurred is done on a single polyacrylamide gel.

In an embodiment of the present invention, the target RNA is modified to comprise a promoter.

In an embodiment of the present invention, the target RNA is modified to comprise a T7 RNA polymerase promoter.

The present invention is also directed to methods of making a catalytically active ribozyme that is specific for a target RNA and accessible to a cleavage site on said target RNA comprising:

(a) identifying a cleavage site on a target RNA using a method as described above;

(b) constructing a ribozyme comprising a sequence that is complementary to a cleavage site of step (a).

The present invention is also directed to catalytically active ribozymes produced by any of the above described methods.

The present invention is also directed to methods of identifying one or more potential sites in a target RNA which are accessible to an antisense oligonucleotide, wherein said method comprises:

(a) generating a library of antisense oligonucleotides, wherein each antisense oligonucleotide of the library comprises regions of random nucleotides flanked by fixed sequences which allow reamplification and transcription;

(b) contacting said target RNA with the library of antisense oligonucleotides;

(c) separating antisense oligonucleotides that bind to said target RNA from antisense oligonucleotides that do not bind;

(d) generating an enriched library of antisense oligonucleotides comprising antisense oligonucleotides bound in step (c);

(e) repeating steps (a) through (d) at least four times to obtain selected antisense oligonucleotides;

(f) sequencing the selected antisense oligonucleotides of step (e); and

(g) comparing the sequences determined in step (f) with the sequence of said target RNA to identify one or more potential sites in said target RNA which are accessible to an antisense oligonucleotide.

The present invention is also directed to methods of making an antisense oligonucleotide that is accessible to a site in a target RNA comprising:

(a) identifying a site in a target RNA using any of the methods described;

(b) constructing an antisense oligonucleotide comprising a sequences that is complementary to a site identified in step (a); wherein said antisense oligonucleotide of step (b) binds to and is accessible to a target RNA.

The present invention is also directed to antisense oligonucleotides made by any of the above-described processes.

The present invention is also directed to methods of conducting real-time PCR comprising labeling any antisense oligonucleotide of the present invention with a detectable label to generate a labeled probe and using said labeled probe in a real-time PCR amplification.

The present invention is also directed to methods of conducting an assay with a fixed polynucleotide array comprising labeling any antisense oligonucleotide of the present invention with a detectable label to generate a labeled probe and using said labeled probe in an assay with a fixed polynucleotide array.

The present invention is also directed to methods of identifying one or more cleavage sites in a target RNA which are accessible to a DNAzyme, said method comprising:

(a) generating a library of DNAzymes, wherein each DNAzyme in said library comprises a catalytically active DNAzyme core, wherein said DNAzyme core is flanked on each side by random nucleotide regions, wherein said random nucleotide regions are limited to no more than seven random nucleotides upstream of said DNAzyme core and no more than eight random nucleotides downstream of said DNAzyme core, wherein said random nucleotide regions are flanked on each side by fixed sequences which allow amplification;

(b) contacting said target RNA with said library of DNAzymes in the absence of Mg²⁺ such that said DNAzyme core is not catalytically active;

(c) separating DNAzymes that bind to said target RNA from DNAzymes that do not bind to said target RNA using a non-denaturing polyacrylamide gel;

(d) generating an enriched library of DNAzymes comprising amplifying by PCR DNAzymes bound in step (c) using two amplification primers, followed by unidirectional PCR amplification using a single primer to generate single stranded DNAzymes;

(e) generating 5′ or 3′ end-labeled target RNA;

(f) contacting said 5′ or 3′ end-labeled target RNA of step (e) with an enriched library of DNAzymes of step (d) under conditions in which said DNAzyme core of said library of DNAzymes is catalytically active such that said target RNA is cleaved to produce cleavage products;

(g) separating said cleavage products from step (f) and determining the sequence or sequences at which cleavage of said end-labeled target RNA occurred as a result of incubation of said end-labeled target RNA with said library of DNAzymes.

The present invention is also directed to methods of making a catalytically active DNAzyme that is specific for a target RNA and accessible to a cleavage site on said target RNA comprising:

(a) identifying a cleavage site on a target RNA using any of the methods herein described;

(b) constructing a DNAzyme comprising a sequence that is complementary to a cleavage site of step (a).

The present invention is also directed to DNAzymes produced by any of the methods of the invention.

This invention provides an improved method of screening a library of nucleic acids to identify cleavage sites of a target RNA. The screening process comprises generating libraries of nucleic acids, including ribozymes, DNAzymes and oligonucleotides. Ribozymes and DNAzymes comprise a catalytic core flanked by random nucleotides. A target RNA is then added to the library of nucleic acids and the nucleic acids that bind to and/or cleave said target RNA are isolated. In one embodiment, the nucleic acids that bind to the target RNA are antisense oligonucleotides.

In a preferred embodiment, the target RNA further comprises a cis-acting catalytic hammerhead ribozyme domain and a 3′ flanking sequence which is reverse complementary to the 3′ end of the particular target RNA so as to impart uniformity in size of the cleaved ribozyme library and to facilitate 3′ end-labeling of the library.

In another preferred embodiment, the nucleic acids in the random pool of nucleic acids further comprises defined sequences 5′ and/or 3′ to the random nucleic acid sequences. In a preferred embodiment, the defined sequences are 10 to 50 nucleotides long. In a more preferred embodiment, the defined sequences are 15 to 20 nucleotides long.

In one embodiment, the target RNA is isolated from an infectious agent. In one embodiment, the target RNA includes, but is not limited to, viral RNA from a single source. The viral RNA may be isolated from pathogenic viral RNAs such as, but not limited to, hepatitis B virus, hepatitis C virus, human immunodeficiency virus, or human papillomavirus.

The invention also encompasses the recombinant nucleic acids encoding the catalytic nucleic acids identified by the screening methods described herein.

### BRIEF DESCRIPTION OF THE DRAWINGS

The foregoing summary, as well as the following detailed description of the invention, will be better understood when read in conjunction with the appended drawings. For the purpose of illustrating the invention, the drawings exhibit embodiment(s) which are presently preferred. It should be understood, however, that the invention is not limited to the precise arrangements and instrumentalities shown.

In the drawings:

FIG. 1. Schematic Representation of the Random Rz Selection Library. (A) Diagram of a hammerhead ribozyme, showing a catalytic core, flanked by 2 random 9 nt 5′/3′-flanking regions. Arrowhead depicts the site of cleavage, just 3′ to the NUH triplet in the target RNA. (B) Procedure for generating the library of random Rz-RNA transcripts. Primers are annealed together, and subjected to PCR amplification to yield a double-stranded DNA library. The T7 RNA polymerase promoter (underlined) is then utilized to transcribe the 48 nt random Rz library; (C) The dsDNA library was generated and sequenced using a PCR-based method, the products were then analyzed on a 6% sequencing gel under standard conditions. The results confirm the presence of the catalytic core and the two random 9 nt regions of the library.

FIG. 2. Schematic Representation of the target RNA. (A) Diagram of a hammerhead ribozyme tail, showing a catalytic core with fixed helix I, a “P”-part that was the 3′-end of target RNA, and a “Q”-part reverse complementary to the P-portion of the target RNA. Arrowhead depicts the site of cleavage, just 3′ to the GUC triplet in the target RNA. (B) Procedure for generating the template of target RNA transcripts. Pre-template, dsDNA generated by PCR/RT-PCR, subjected to PCR amplification with 5′/3′-end primers to yield a double-stranded DNA library. The T7 RNA polymerase promoter (underlined) is then utilized to transcribe the target RNA with Rz tail; (C) Target RNA with a precise 3′-end was self-liberated during in vitro transcription, the transcripts were then analyzed on a 6% sequencing gel under standard conditions.

FIG. 3. Schematic overview of the library selection procedure. The Rz-library RNA and target RNA are annealed to form RNA-RNA complex (Panel A.a). The complexes are then isolated (Panel A.b) and regenerated (Panel A.c) by RT-PCR and in vitro transcription. The re-amplified Rz-library RNA and the 5′ or 3′-end 32P-labeled target RNA in presence of magnesium (Panel A.d), and then are mixed to initiate Rz-catalytic activity (Panel A.e). Finally, the cleaved products are separated on a 6% sequencing gel under standard conditions (Panel A.f and Panel B). In Panel B, lanes 1 and 2 are the target RNA incubated with random and selected Rz-library RNA (respectively), lanes 3 and 4 are G and A hydrolysis ladders generated from target RNA by RNase T1 and U2 digestions (respectively). The positions of the major cleavage products is shown to the right.

FIG. 4. In vitro Cleavage Analyses of HPV16-E6/E7 Targeted Rz. The numbers of the 11 Rzs tested represent the locations of the cleavage sites within the HPV16-E6/E7 transcript. Individual Rz were transcribed in vitro from double-stranded DNA templates as described, and mixed with 5′-end 32P-labeled HPV16 E6/E7 target RNA (782 nt). Incubations were for 30 min at 37° C. in 20 mM Tris-HCl (pH 7.4), 5 or 25 mM MgCl2. Following cleavage, the products were separated by denaturing PAGE, and results were quantitated using a Phosphor-Imager. The size of the cleavage products is shown to the right, and the activities of the sRz are shown at the bottom, relative to sRz427, which was the most active sRz identified using our modified SELEX library selection procedure (Pan et al 2001).

FIG. 5. In vitro Cleavage Analyses of HPV16-E6/E7 Targeted sRz. 100 mM of the individual sRz and 10 nM of 5′-end 32P-labeled HPV16 target RNA (782 nt) were incubated for 30 min at 37° C. in 20 mM Tris-HCl (pH 7.4), also containing 1, 5 or 25 mM MgCl2. Following cleavage, the products were separated by denaturing PAGE, the gel was dried and subjected to autoradiography.

FIG. 6. In vitro Cleavage Analyses of HPV16-E6/E7 Targeted sRz59. 1, 2.6, 6.4, 16, 40 and 100 nM of sRz59 and 10 nM of 5′-end 32P-labeled HPV16 target RNA (782 nt) were incubated for 30 min at 37° C. in 20 mM Tris-HCl (pH 7.4), also containing 5 or 25 mM MgCl2. Following cleavage, the products were separated by denaturing PAGE, the gel was dried and subjected to autoradiography.

FIG. 7. Diagrammatic Representation of SNIP. The CLIP and CHOP portions of the SNIP cassette are shown beneath the upper diagram. Depicted is a double internal Rz (dITRz), and the various 3′-modifications are liberated with these trans-acting ribozymes. The sites of autocatalytic cleavage are designated S1-S4, and their positions are marked with arrows on the lower diagram. The size of the respective nonfunctional regions of the processed cassettes are shown for the various versions (in nt).

FIG. 8. Autocatalytic Processing of SNIP cassettes. (A) In Vitro Autocatalytic Processing of a SNIPAARz cassette. SNIPAARz777/885, targeted to Hepatitis B Virus, was selected as a representative example. In vitro transcription reactions were run for 0, 5, 10, 20, or 60 minutes, after which reactions were terminated. Reaction products were examined by PAGE on 8% sequencing gels, the gels were dried, and examined by autoradiography. Nucleotide (nt) sizes are shown to the left. Position of the liberated dITRz is indicated with the double arrows; dITRz differ slightly in size by design when liberated from the CLIP or CHOP portions of the cassette. As is evident, autocatalytic processing proceeds very efficiently in vitro. (B) Real-Time RT/PCR Quantitation of SNIP Cassettes within Cells. 293T Cells were transfected with the various SNIPRz777/885 cassettes, and RNA was harvested 24 h later. Various primer pairs were used to amplify the respective regions of the cassettes. Upper blue lines represent amplification cycles for the SNIP regions, whereas the lower red lines represent amplification of 18S rRNA. Volume differences were minimal and no corrections were required.

FIG. 9. Stability of the dITRz liberated from the various SNIP cassettes. (A) Stability of liberated dITRz within cells. 293T Cells were transfected with the various SNIP cassettes containing the dITRz Rz777/885 in both CLIP and CHOP sites. 48 h later, RNA was harvested and quantitative RT/PCR was performed. Products were separated by PAGE on 8% gels, and analyzed by autoradiography. Relative concentrations of the dITRz, compared with that from the SNIP cassette (no 3′-end modifications) were 2.6×, 2.5×, and 1.5× for the SNIPAA, SNIPHIS, and SNIPHP cassettes, respectively. The same values were obtained using 2 different primer pairs. The negative control sample had been transfected with a GFP construct. (B) Effects of 3′-End Modifications on catalytic activity in vitro. The dITRz Rz777/885 in the various SNIP cassettes were tested in vitro with the corresponding HBV target RNA for 7, 20, or 60 minutes (as indicated) at 37° C. in the presence of 5 mM MgCl₂. Products were then separated by PAGE in 6% gels, and the products analyzed by autoradiography. The catalytic activity of the dITRz liberated from the SNIPAA cassette was 130% of the control activity. dITRz activities from the SNIPHP cassette were the same as the control activity, while that from the SNIPHIS cassette was decreased approximately 20%.

FIG. 10. Reduction of HPV16 E6/E7Target RNA in co-transfection experiments. 293T cells were co-transfected with plasmids encoding the HPV16 E6/E7 mRNA and the SNIPAAsRz constructs as indicated. After 3 and 5 days, RNA was isolated and E6/E7 transcript was quantitated by radiolabeled RT/PCR. A portion of 18S rRNA was amplified concurrently as a standard.

FIG. 11. Real-time PCR using HPV 11 template.

FIG. 12. Procedure for 3′-³²P end labeling of target RNAs. Diagram shows the addition of a 3′ cis-acting hammerhead ribozyme to a target RNA. Nucleotides N1-N10 represent ten nucleotides at the 3′ end of a target RNA. The arrow depicts the site of cleavage. X1-X10 are chosen so as to be reverse complementary to N1-N10 (i.e., X1 is complementary to N10, X2 is complementary to N9, etc.). The starred nucleotides are added, if necessary, to provide the nucleotide triplet cleavage site. If the target RNA ends in a “G”, then a T and C are added. If an RNA ends in a GT, only a C is added, and if it ends in a GTC, these nucleotides are not added. XbaI denotes an XbaI restriction endonuclease site, which is added for cloning purposes.

The purpose of this procedure is to add a catalytic hammerhead ribozyme domain and a 3′ flanking sequence of 10 nt which is reverse complementary to the 3′ end of the particular target RNA undergoing library selection. This procedure produces transcripts with a precisely defined 3′-end. Otherwise, the RNA polymerases do not precisely terminate at a given nucleotide, producing a family of transcripts which differ in length by 1, 2 or a few nucleotides, precluding identification of cutting sites on sequencing gels.

FIG. 13. Schematic overview of the oligonucleotide-library selection procedure. The ssDNA-oligonucleotide library is converted to dsDNA form (A), and used to transcribe the oligonucleotide-guide RNA library (B). The target RNA is then mixed with the oligonucleotide library, and the bound pool is isolated by PAGE under nondenaturing conditions (C). The bound oligonucleotide pool is then isolated, converted into cDNA (D), which is again converted into dsDNA (E), which constitutes a “round” of selection. After n rounds of selection, the dsDNA pool is cloned, and many representative sequences are obtained, matched to the target sequence, and Rz are designed against the identified sites and tested for catalytic activity in vitro.

### DETAILED DESCRIPTION OF THE INVENTION

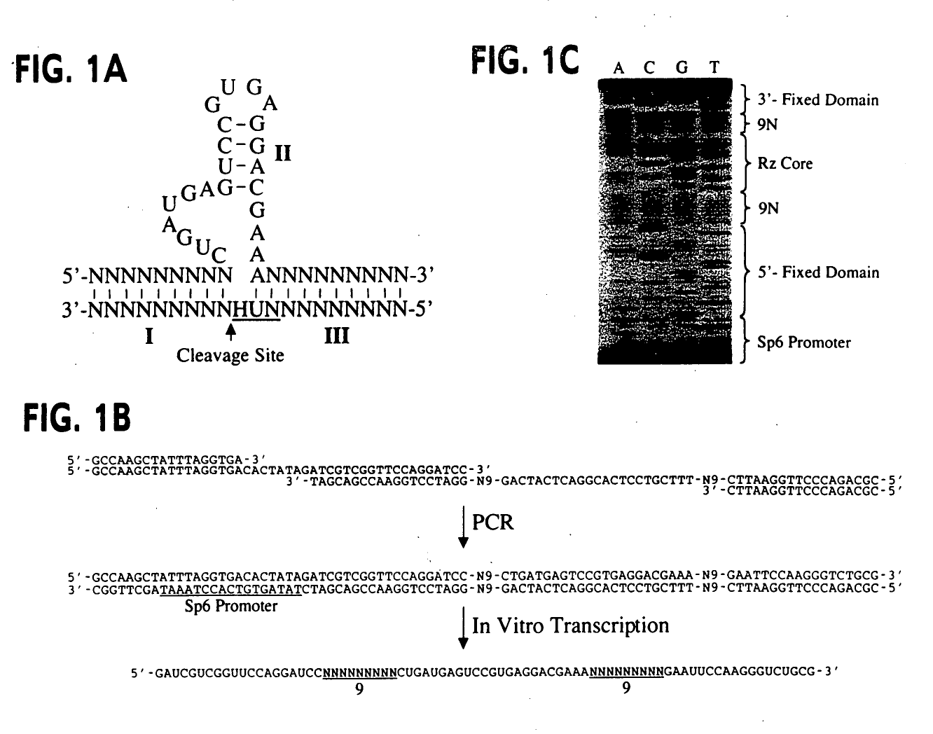

Ribozymes are catalytic RNA molecules with endoribonuclease activity. They are able to catalyze the irreversible site-specific cleavage-reaction of multiple transcripts in the presence of a divalent metal ion, typically magnesium, to yield products with 5′-hydroxyl and 2′ 3′-cyclic phosphate termini (Gaughan & Whitehead 1999, James & Gibson 1998, Lilley 1999). The “hammerhead ribozyme” (Rz), one of the smallest types of ribozyme, was derived from self-cleaving plant viral RNAs (Symons 1992), and is the most widely employed for inhibiting the function(s) of target genes (Amarzguioui & Prydz 1998, Birikh et al 1997b, Jen & Gewirtz 2000, Sun et al 2000). Functional Rz can be designed to target transcripts in trans by generating RNA molecules with complementary sequences in the helix I and helix III regions that flank a helix II catalytic core (FIG. 1A). The major sequence constraint in the target RNA is the presence of a cleavable 5′-NUH-3′ triplet (where N represents any nucleotide and H represents A, C, or U). Complementary sequences confer specificity and generally constitute 14-16 nucleotides in total, extending on both sides of the target site selected. This allows sufficient specificity for the cleavage reaction while allowing ready dissociation from the target, which is typically the rate limiting step in the catalytic cycle (Goodchild & Kohli 1991, Hendry & McCall 1996, Parker et al 1992).

Since the consensus triplet sequence is not very restrictive, virtually any mRNA or viral genomic RNA of interest is likely to possess numerous potential target sites for Rz cleavage. To date, most investigators have selected target sites through a computer-aided process that searches for single-stranded regions that contain a suitable nucleotide triplet. Unfortunately, possibly due to RNA secondary and tertiary structure preventing binding of the ribozymes to their targets, it is frequently observed that only a small fraction of Rz designed in this manner produce significant reductions in target RNA levels within cells (Benedict et al 1998, Crone et al 1999, Eldadah et al 2000, Folini et al 2000, Macejak et al 2000, Passman et al 2000, Perlman et al 2000, Ren et al 1998, Salmi et al 2000, Suzuki et al 2000). Indeed, target site selection seems to constitute the major problem in designing Rz with optimal activity, especially with long target transcripts.

Birikh et al. (Birikh et al 1997a) used a completely randomized oligonucleotide (dN10) in conjunction with RNase H to map sites that are accessible for oligonucleotide binding in an RNA transcript: the best Rz generated in this fashion was 150-fold more active than the most efficient Rz designed on the basis of the mFold program (Zuker & Stiegler 1981). In a previous study, a modified SELEX method was used to locate accessible sites within any targeted RNA by systematically isolating guide-RNAs from a large pool of random RNA sequences (Pan et al 2001). 50% of Rz designed to cleave the identified accessible sites were highly active in cleaving their long, structured targets, with Kcat/Km values of around 106 (M⁻¹ min⁻¹). A Rz to human hepatitis B virus effectively inhibited viral replication and secretion in cell culture (Pan et al 2001).

Although the experimental approaches for identification of accessible binding sites (association step, Km) offer clear advantages, the above methods could determine neither the actual Rz-cleavage site, nor the availability of nucleotides near the cleavage site; if accessibility is limited, this could result in decreased activity (chemical step, Kcat; (Campbell et al 1997, Clouet-d'Orval & Uhlenbeck 1997). Lieber & Strauss (Lieber & Strauss 1995) constructed a Rz library that was targeted to a pre-selected triplet, and contained randomized sequences in the annealing arms, which allowed the screening of suitable sites in the target-RNA molecule. In this case, the selected Rz cleaved target transcripts efficiently in vitro and inhibited gene expression in cell culture (Lieber & Kay 1996). This procedure identified both the accessible sites and the precise position of cleavable triplets along with available nucleotides, but the results were biased because reverse transcription, tailing and PCR were necessary to amplify the cleavage products. More importantly, cloning and sequencing procedures were required, as they were with our modified SELEX method (Pan et al 2001).

The present invention is directed to methods of identifying cleavage sites in a target RNA which are accessible to catalytic nucleic acids such as ribozymes or DNAzymes.

In one embodiment, the methods of identifying cleavage sites in a target RNA involve generating a library of RNAs wherein each RNA in the library comprises a catalytic core that is a hammerhead ribozyme. The catalytic core of the ribozyme is flanked by a random sequence of nucleotides that is preferably between about six to about twelve nucleotides in length, more prefereably between about seven to about eleven nucleotides in length, more preferably between about eight to about ten nucleotides in length and most preferably about nine nucleotides in length. The random sequences flanking the catalytic core are flanked, in turn, by fixed sequences that allow for amplification of the RNA by PCR as well as a sequence or sequences that allow transcription of the RNA. In a preferred embodiment, the sequence allowing transcription of the RNA is an SP6 promoter.

In an embodiment, the fixed sequences that allow for PCR amplification are about 7, 8, 9, 10, 11, 12, 13, 14, 15, 16, 17, 18 or 19 nucleotides in length; in an embodiment the fixed sequences that allow for PCR amplification are about 20, 21, 22, 23, 24, 25, 26, 27, 28, or 29 nucleotides in length; in an embodiment the fixed sequences that allow for PCR amplification are about 30, 31, 32, 33, 34, 35, 36, 37, 38, or 39 nucleotides in length; in an embodiment, the fixed sequences that allow for PCR amplification are about 40, 41, 42, 43, 44, 45, 46, 47, 48 or 49 nucleotides in length; in an embodiment, the fixed sequences that allow for PCR amplification are about 50, 51, 52, 53, 54, 55, 56, 57, 58, 59 or 60 nucleotides in length.

The methods of identifying cleavage sites in a target RNA of the present invention further comprise contacting a target RNA with a library of RNA molecules, as described in the previous paragraphs, wherein the library of RNA molecules and the target RNA are combined under conditions wherein the ribozyme core of the library RNA molecules is catalytically inactive. In a preferred embodiment, these conditions are in the absence of Mg²⁺. The methods of the present invention further comprise separating RNA molecules that do bind to the target RNA from RNA molecules that do not bind to the target RNA. In a preferred embodiment, the separation is done using gel chromatography, such as, for example, polyacrylamide gel electrophoresis (PAGE) chromatograpy, or by column chromatography or by HPLC.

The methods of identifying cleavage sites in a target RNA of the present invention further comprise generating an enriched library of RNA molecules that bind to a target RNA. In a preferred embodiment, the enriched library of RNA molecules that bind to a target RNA molecule is generated by PCR amplification of the RNA molecules that bound to the target RNA.

In a preferred embodiment, the steps of contacting the library of RNA molecules with the target RNA under conditions in which the ribozyme catalytic core of the RNA molecules is inactive, separating RNA that binds to the target RNA from RNA that does not bind to the target RNA and generating an enriched library of RNAs is repeated at least one additional time with a reduced ratio of target RNA to library of RNA. In another embodiment, these steps are repeated at least two additional times with a reduced ratio of target RNA to library of RNA. In another preferred embodiment, these steps are repeated at least three additional times with a reduced ratio of target RNA to library of RNA. In another preferred embodiment, these steps are repeated at least four additional times with a reduced ratio of target RNA to library of RNA.

The methods of identifying cleavage sites in a target RNA further comprise generating a 5′ or a 3′ end-labeled target RNA and contacting this target RNA with an enriched library of RNA under conditions in which the ribozyme core of the enriched library of RNAs is catalytically active such that the labeled target RNA is cleaved to produce cleavage products and the sequence of the cleavage site is determined.

In a further embodiment, methods of identifying cleavage sites in a target RNA further comprise using a 3′ end-labeled target RNA that is of uniform length that is produced by constructing a target RNA comprising a 3′ cis-acting catalytic ribozyme having a 3′ flanking sequence that is reverse complementary to the 3′ end of the target RNA wherein the target RNA is cleaved at its 3′ end by the 3′ cis-acting catalytic ribozyme. This produces a target RNA of uniform length that is then labeled at the 3′ end.

The present invention is also directed to methods of making catalytic nucleic acids such as ribozymes or DNAzymes by designing them based on the cleavage sites identified using the methods of the previous paragraphs. The present invention is also directed to catalytic nucleic acids such as ribozymes and DNAzymes that are made using the disclosed methods.

The invention is also directed to methods of conducting real-time PCR using probes designed based on the accessible cleavage sites identified using the methods described herein. The invention is also directed to methods of conducting assays using fixed polynucleotide arrays using probes designed based on the accessible cleavage sites identified using the methods described herein.

### Oligonucleotide Libraries

The oligonucleotide selection technique has been refined in terms of the design of the library of random sequences, taking into account data on catalytic activity and specificity, and employing it to determine accessible sites on target RNAs. In one embodiment, the number of nucleotides present in the random sequence may have about nine random nucleotides upstream of a central catalytic core, followed by about six random nucleotides downstream of the central catalytic core. In a preferred embodiment, the catalytic core is a central TC.

In contrast, another method known in the art (e.g., Lieber & Strauss, 1995, Mol Cell Biol 15:540-551), utilizes 13 random nucleotides upstream of a central catalytic core, followed by 11 random nucleotides, and the target site nucleotide triplet is more restrictive. The advantage of limiting the number of random nucleotides is the increased accessibility to the cleavage site in the target RNA. By using a procedure that increases accessibility to the cleavage sites, active target sites may be determined throughout relatively long transcripts. Importantly, the Lieber and Strauss method does not employ a SELEX procedure for reamplification and repetitive binding. This is in contrast to the methods of the present invention.

The target RNA may further comprise a catalytic hammerhead ribozyme domain and a 3′ flanking sequence which is reverse complementary to the 3′ end of the particular target RNA. The addition of a catalytic hammerhead ribozyme to the 3′ end of the target RNA enables target RNA to be ³²P-labeled at the 3′ end. The addition of a cis-acting hammerhead ribozyme sequence to the target RNA produces a precise 3′ end of the target RNA. This addition allows identification of sites closer to the 3′ end, since otherwise microheterogeneity of polymerase termination at the 3′ end precludes direct 3′ end labeling. Prior to end-labeling, the cyclic phosphate bond of the 3′-terminal C is broken by incubating the RNA in 10 mM HCl at 25° C. for 4 hours. The RNA is then labeled with ³²P-CoTP using poly(A) polymerase.

The 3′ end of the target RNA described above may be labeled with any detectable marker, using methods for labeling known in the art. A “detectable marker” refers to a moiety, such as a radioactive isotope or group containing same, or nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent).

### Ribozyme (Rz) Libraries

The Rz library selection procedures of the present invention have been modified from previously described methods. The methods of the present invention take into account data on catalytic activity and specificity to determine accessible target sites. The ribozymes identified using the present methods are distinguished from ribozymes designed using oligonucleotide libraries because the ribozymes of the present invention have a greater activity than those designed using oligonucleotide libraries.

In one embodiment, the number of nucleotides present in the random sequence in the RNA library has about nine random nucleotides upstream of a central catalytic core, followed by about six random nucleotides downstream of the central catalytic core. In contrast, another method known in the art (e.g., Lieber & Strauss, 1995, Mol Cell Biol 15:540-551), utilizes 13 random nucleotides upstream of a central catalytic core, followed by 11 random nucleotides, and the target site nucleotide triplet is more restrictive. The advantage of limiting the number of random nucleotides is the increased accessibility to the cleavage site in the target RNA. By using a procedure that increases accessibility to the cleavage sites, active target sites may be determined throughout relatively long transcripts.

The target RNA may further comprise a catalytic hammerhead ribozyme domain and a 3′ flanking sequence which is reverse complementary to the 3′ end of the particular target RNA. The addition of a catalytic hammerhead ribozyme to the 3′ end of the target RNA enables target RNA to be ³²P-labeled at the 3′ end. The addition of a cis-acting hammerhead ribozyme sequence to the target RNA produces a precise 3′ end of the target RNA. This addition allows identification of sites closer to the 3′ end, since otherwise microheterogeneity at the 3′ end precludes direct 3′ end labeling. Prior to end-labeling, the cyclic phosphate bond of the 3′-terminal C is broken by incubating the RNA in 10 mM HCl at 25° C. for 4 hours. The RNA is then labeled with ³²P-CoTP using poly(A) polymerase.

The 3′ end of the target RNA described above may be labeled with any detectable marker, using methods for labeling known in the art. A “detectable marker” refers to a moiety, such as a radioactive isotope or group containing same, or nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent).

The invention also includes ribozymes wherein the catalytic core is flanked by random nucleotides. In a preferred embodiment, the ribozyme is a hammerhead ribozyme.

The invention also comprises ribozymes which are triple ribozymes. In one embodiment, the triple ribozyme is a ribozyme cassette comprising cis-acting ribozymes flanking a trans-acting ribozyme that cleaves said target RNA. Such triple ribozymes are described in U.S. Pat. No. 5,824,519 and PCT Publications WO 97/17433, WO 98/24925, WO 99/67400, and WO 00/61804, which are incorporated herein by reference in their entireties. In a preferred embodiment, the ribozyme cassette is CLIP. In this preferred embodiment, the two cis-acting ribozymes function to release themselves from the primary transcript, liberating the trans-acting internal ribozyme with minimal non-specific flanking sequences. In another embodiment, the ribozyme is SNIP or SNIPAA.

The invention also encompasses the recombinant nucleic acids encoding the ribozymes elucidated from the screening methods described herein.

### DNAzyme (Dz) Libraries

The present invention is also directed to DNAzyme library selection procedures. The methods of the present invention take into account data on catalytic activity and specificity to determine accessible target sites. The DNAzymes identified using the present methods are distinguished from DNAzymes designed using oligonucleotide libraries because the DNAzymes of the present invention have a greater activity than those designed using oligonucleotide libraries.

Specifically, in the construction of the DNA library, the number of random nucleotides present in the random sequence has been limited to no more than seven random nucleotides upstream of a central catalytic core, followed by no more than eight random nucleotides downstream of the central catalytic core. In a preferred embodiment, the catalytic core is no more than 15 nucleotides.

The target RNA may further comprise a catalytic hammerhead ribozyme domain and a 3′ flanking sequence which is reverse complementary to the 3′ end of the particular target RNA. The addition of a catalytic hammerhead ribozyme to the 3′ end of the target RNA enables target RNA to be ³²P-labeled at the 3′ end. The addition of a cis-acting hammerhead ribozyme sequence to the target RNA produces a precise 3′ end of the target RNA. This addition allows identification of sites closer to the 3′ end, since otherwise microheterogeneity of polymerase termination at the 3′ end precludes direct 3′ end labeling. Prior to end-labeling, the cyclic phosphate bond of the 3′-terminal C is broken by incubating the RNA in 10 mM HCl at 25 C for 4 hours. The RNA is then labeled with ³²P-CoTP using poly(A) polymerase.

The 3′ end of the target RNA described above may be labeled with any detectable marker, using methods for labeling known in the art. A “detectable marker” refers to a moiety, such as a radioactive isotope or group containing same, or nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent).

The invention also encompasses the recombinant nucleic acids encoding the DNAzymes elucidated from the screening methods described herein.

### Eucaryotic and Procaryotic Expression Vectors

The present invention encompasses expression systems, including both eucaryotic and procaryotic expression vectors, which may be used to express the catalytic nucleic acids of the invention. The DNA expression vectors and viral vectors containing the catalytic nucleic acids of the present invention may be produced by recombinant DNA technology using techniques well known in the art. Thus, methods for preparing the expression vectors and viral vectors of the invention for expressing the catalytic nucleic acids are described herein. Methods which are well known to those skilled in the art can be used to construct expression vectors containing gene product coding sequences and appropriate transcriptional and translational control signals. These methods include, for example, in vitro recombinant DNA techniques, synthetic techniques, and in vivo genetic recombination. See, for example, the techniques described in Sambrook et al., Molecular Cloning: A Laboratory Manual, 2nd Ed., Cold Spring Harbor Laboratory, Cold Spring Harbor, N.Y., 1989.

A variety of host-expression vector systems may be utilized to express the selected catalytic nucleic acids of the invention. These include but are not limited to microorganisms such as bacteria transformed with recombinant bacteriophage DNA, plasmid DNA or cosmid DNA expression vectors containing the catalytic nucleic acids; yeast (e.g., Saccharomyces, Pichia) transformed with recombinant yeast expression vectors containing the catalytic nucleic acids; insect cell systems infected with recombinant virus expression vectors (e.g., baculovirus) containing the catalytic nucleic acids; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the catalytic nucleic acids; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

### Delivery and Expression by Viral Vectors

In accordance with the present invention, a wide variety of viruses and viral vectors may be used to deliver the nucleotide sequences encoding the catalytic nucleic acids of the present invention, a few examples of which are described below. In this regard, a variety of viruses may be genetically engineered to transcribe the selected catalytic nucleic acids in order to target a specific pathogen.

The present invention also relates to the delivery of the catalytic nucleic acids of the invention to cell or pathogen by abiologic or biologic systems. In a specific embodiment, a catalytic nucleic acid of the invention is delivered to a bacterial cell by a bacteriophage capable of infecting a pathogenic bacteria. In a further embodiment, bacteriophage are selected for their ability to infect a particular species of bacteria, and are used to deliver a catalytic nucleic acid for the eradication of such bacterial species from a host.

The invention provides for use of a virion which can also be any bacteriophage which specifically infects a bacterial pathogen of the present invention as well as any virus which can be specifically targeted to infect the pathogen of the present invention. For example, the bacteriophage can include, but is not limited to, those specific for bacterial cells of the following genera: Bacillus, Campylobacter, Corynebacterium, Enterobacter, Enterococcus, Escherichia, Kleosiella, Mycobacterium, Pseudomonas, Salmonella, Shigella, Staphylococcus, Streptococcus, Vibrio, Streptomyces, Yersinia and the like (see, e.g., the American Type Culture Collection Catalogue of Bacteria and Bacteriophages, latest edition, Rockville, Md.), as well as any other bacteriophages now known or later identified to specifically infect a bacterial pathogen of this invention. The invention also provides for the use of a virion which specifically infects a fungal pathogen.

This delivery system consists of a DNA plasmid carrying the nucleic acids coding for the catalytic nucleic acids packaged into viral particles. Specificity is conferred by the promoter driving transcription of the catalytic nucleic acids and by the host specificity of the viral vehicle. Specificity is also conferred by the origin of replication controlling vector replication.

In the virions of the present invention, the non-viral DNA can encode the catalytic nucleic acids. The non-viral DNA can further comprise a pathogen-specific or tissue-specific promoter operably linked to a sequence encoding one or more catalytic nucleic acids.

Abiologic delivery of catalytic nucleic acids is accomplished by a variety of methods, including packaging plasmid DNA carrying the gene(s) that codes for the catalytic nucleic acids into liposomes or by complexing the plasmid DNA carrying the gene(s) that codes for the catalytic nucleic acids with lipids or liposomes to form DNA-lipid or DNA-liposome complexes. The liposome is composed of cationic and neutral lipids commonly used to transfect cells in vitro. The cationic lipids complex with the plasmid DNA and form liposomes. The liposome delivery system of the invention can be used to deliver a catalytic nucleic acid of the invention.

Cationic and neutral liposomes are contemplated by this invention. Cationic liposomes can be complexed with a negatively-charged biologically active molecule (e.g., DNA) by mixing these components and allowing them to charge-associate. Cationic liposomes are particularly useful when the biologically active molecule is a nucleic acid because of the nucleic acids negative charge. Examples of cationic liposomes include lipofectin, lipofectamine, lipofectace and DOTAP (Hawley-Nelson et al., 1992, Focus 15(3):73-83; Felgner et al., 1987, Proc. Natl. Acad. Sci. U.S.A. 84:7413; Stewart et al., 1992, Human Gene Therapy 3:267-275). Procedures for forming cationic liposomes encasing substances are standard in the art (Nicolau et al., 1987, Methods Enzymol. 149:157) and can readily be utilized herein by one of ordinary skill in the art to encase the complex of this invention.

In yet another embodiment of the present invention, the plasmid DNA carrying the gene(s) that codes for the catalytic nucleic acids of the invention are complexed with liposomes using an improved method to achieve increased systemic delivery and gene expression (Templeton et al., 1997, Nature Biotechnology 15: 647-652, incorporated herein by reference in its entirety). The present invention is also directed to an improved formulation of cationic lipids which greatly increases the efficiency of DNA delivery to host cells, with extended half-life in vivo and procedures to target specific tissues in vivo. For example, but not by limitation, peptides and proteins may be engineered for incorporation into the outer lipid bilayer, such as liver-specific proteins which leads to substantially enhanced delivery to the liver etc.

In one embodiment of the present invention, systemic delivery and in vivo and ex vivo gene expression is optimized using commercially available cationic lipids, e.g., dimethyldioctadeclammonium bromide (DDAB); a biodegradable lipid 1,2-bis(oleoyloxy)-3-(trimethylammonio) propane (DOTAP); these liposomes may be mixed with a neutral lipid, e.g., L-* dioleoyl phosphatidylethanolamine (DOPE) or cholesterol (Chol), two commonly used neutral lipids for systemic delivery. DNA:liposome ratios may be optimized using the methods used by those of skill in the art (e.g., see Templeton et al., 1997, Nature Biotechnology 15: 647-152, incorporated herein by reference in its entirety).

In yet another embodiment of the present invention, the plasmid DNA carrying the nucleic acids encoding the catalytic nucleic acids of the invention may be delivered via polycations, molecules which carry multiple positive charges and are used to achieve gene transfer in vivo and ex vivo. Polycations, such as polyethylenimine, may be used to achieve successful gene transfer in vivo and ex vivo (e.g., see Boletta et al., 1996, J. Am. Soc. Nephrol. 7: 1728, incorporated herein by reference in this entirety.)

The liposomes may be incorporated into a topical ointment, cream, gel or solution for application or delivered in other forms, such as a solution which can be injected into an abscess or delivered systemically, or delivered by an aerosol.

### Arrays

In addition, gene expression assays using gene expression arrays or microarrays or fixed polynucleotide arrays are now available for identifying changes in gene expression patterns between different cells or tissue types, i.e., as a diagnostic tool (see, e.g., Schena et al., 1995, Science 270:467-470; Lockhart et al., 1996, Nature Biotechnology 14:1674-1680; and Blanchard et al., 1996, Nature Biotechnology 14:1649). Thus, in another, alternative embodiment of the invention, the nucleic acids identified by the methods of the invention described herein may be arrayed on a gene expression array or microarray and utilized for identifying changes in gene expression pattern, including applications for diagnostic purposes. In a preferred embodiment, the nucleic acids are antisense oligonucleotides.

### Real-Time or Quantitative PCR

Quantitative real-time polymerase chain reaction (PCR) is a relatively new technology that provides a broad dynamic range (at least five orders of magnitude) for detecting specific gene sequences with excellent sensitivity and precision. DNA and RNA can be quantified using this detection system without laborious post-PCR processing. Quantitative real-time PCR is based on detection of a fluorescent signal produced proportionally during the amplification of a PCR product. The chemistry is the key to the detection system. A probe (ie, TaqMan) is designed to anneal to the target sequence between the traditional forward and reverse primers. The probe is labeled at the 5′ end with a reporter fluorochrome (such as, for example, 6-carboxyfluorescein [6-FAM]) and a quencher fluorochrome (6-carboxy-tetramethyl-rhodamine [TAMRA]) added at any T position or at the 3′ end. The probe is usually designed to have a higher Tm than the primers, and during the extension phase, the probe should be 100% hybridized for success of the assay. As long as both fluorochromes are on the probe, the quencher molecule stops all fluorescence by the reporter. However, as Taq polymerase extends the primer, the intrinsic 5′ to 3′ nuclease activity of Taq degrades the probe, releasing the reporter fluorochrome. The amount of fluorescence released during the amplification cycle is proportional to the amount of product generated in each cycle. Additional details regarding Real-time or quantitative PCR may be found, for example in the following publications, each of which is hereby incorporated by reference in its entirety: Gibson U E M, Heid C A, Williams P M. A novel method for real-time quantitative RT-PCR. Genome Res 1996;6:995-1001; Heid C A, Stevens J, Livak K J, Williams P M. Real-time quantitative PCR. Genome Res 1996; 6:986-994; Livak K J, Flood S J A, Marmaro J, Giusti W, Deetz K. Oligonucleotides with fluorescent dyes at opposite ends provide a quenched probe system useful for detecting PCR product and nucleic acid hybridization. PCR Methods Appl 1995;4:357-362; Holland P M, Abramson R D, Watson R, Gelfand D H. Detection of specific polymerase chain reaction product by utilizing the 5′-3′ exonuclease activity of Thermus aquaticus DNA polymerase. Proc Natl Acad Sci USA 1991;88:7276-7280.

Additional details regarding the invention for selecting catalytic nucleic acids may be found in U.S. Pat. No. 5,824,519 and PCT Publications WO 97/17433, WO 98/24925, WO 99/67400, and WO 00/61804, which are incorporated herein by reference in their entireties.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

Without further description, it is believed that a person of ordinary skill in the art can, using the preceding description and the following illustrative examples, make and utilize the present invention and practice the disclosed methods. The following working examples therefore, specifically point out the preferred embodiments of the present invention, and are not to be construed as limiting in any way the remainder of the disclosure.

### EXAMPLES

### Example 1

### A Selection System for Identifying Ribozyme Target Cleavage Site Accessibility

Results and Discussion

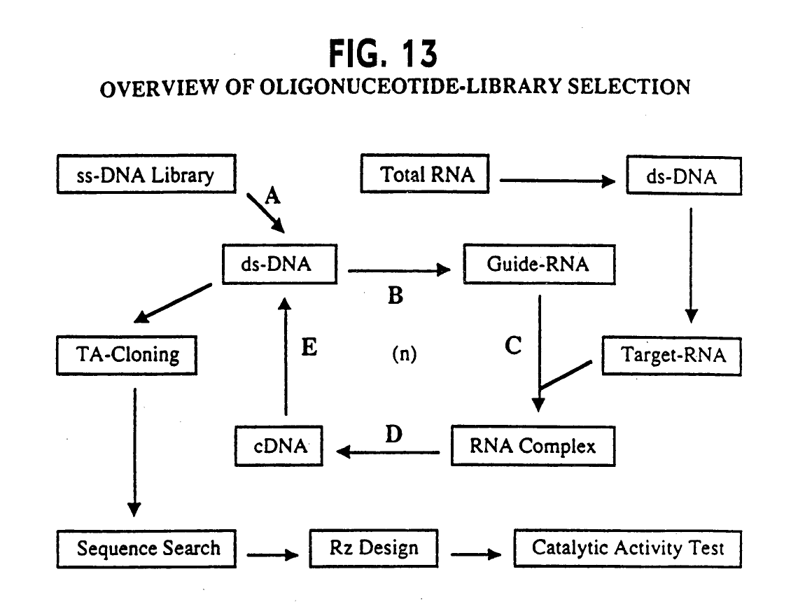

A double-stranded DNA library was used to generate a guide-RNA library (which is a library of RNA oligonucleotides) with multiple copies of approximately 10⁹ different sequences. Each transcript was 48 nt long, with a central GA flanked by 6Ns/9Ns and defined 5′/3′-ends. The guide-RNA library was subjected to selection with each of 3 different target RNAs (HBV, Pol I, and PTEN) under physiological conditions, to isolate RNA molecules that bound the corresponding target-RNA (FIG. 13). Other target RNAs that have been used include HPV E6/E7 and Sf1. The isolated bound guide-RNA pool was subsequently amplified and subjected to another round of selection at a lower target-RNA concentration to increase the selection stringency. Multiple rounds of selection and amplification resulted in an exponential increase of the best binding guide-RNA transcripts. Compared with the unselected guide-RNA library, the 4-round selected guide-RNA pool (i.e., that obtained after 4 rounds of binding and reamplification) for HBV target-RNA had an increased target binding affinity of almost 3000-fold at a concentration of 25 nM. This same HBV-selected guide-RNA pool showed a minimal increase in binding affinity when allowed to hybridize to a non-target RNA such as PTEN RNA. After 5 rounds of selection, the binding affinity of HBV selected guide-RNA pool to HBV target-RNA reached its highest level: it showed 3800-fold higher affinity than the random guide-RNA pool, and slightly higher (1.3-fold) affinity than the 6 round selected guide-RNA pool bound with HBV target-RNA. This presumably reflects saturation of available sites.

PCR products generated from the 5-round selected RNA pools were cloned and sequenced (screening was performed on 26, 32, and 37 clones for HBV, Pol I, and PTEN, respectively), and the sequences were analyzed using the MacVectorTM 5.0 program (Table 1). About 50% of the obtained sequences clustered at 3-5 specific regions of the corresponding target-RNAs while an additional 28% were scattered throughout the target. These sequences define a number of potential cleavage sites for Rz targeting. Another 22% of the obtained sequences did not match sites within their respective target RNAs; they were presumably isolated due to structural affinity or non-specific effects and not via base-pairing interactions. It is likely that more stringent annealing conditions might reduce binding of the non-specific sequences.

TABLE 1
Summary of selected guide-RNA and Rz cleavage site.

| Target | Location | No. of guide-RNA | % | Selected Rz |
|---|---|---|---|---|
| HBV | 880-908 | 8 | 30 | sRz-885 |
| | 460-472 | 2 | 7.7 | sRz-469 |
| | 808-827 | 2 | 7.7 | |
| | scattered | 7 | 26.9 | sRz-408/777 |
| | unmatched | 7 | 26.9 | |
| Pol I | 445-458 | 7 | 21.9 | sRz-458 |
| | 339-359 | 4 | 12.5 | sRz-353 |
| | 589-602 | 3 | 9.4 | sRz-595 |
| | 60-76 | 2 | 6.2 | sRz-70 |
| | scattered | 8 | 25 | |
| | unmatched | 8 | 25 | |
| PTEN | 277-293 | 6 | 16.2 | sRz-281 |
| | 673-687 | 5 | 13.5 | sRz-681 |
| | 692-705 | 3 | 8.1 | |
| | 1057-1072 | 3 | 8.1 | |
| | 6-19 | 2 | 5.4 | |
| | scattered | 12 | 32.4 | sRz-425/499/774 |
| | unmatched | 6 | 16.2 | |

| | Clustered | Scattered | Unmatched |
|---|---|---|---|
| No. of guide-RNA | 47 | 27 | 21 |
| % | 49.5 | 28.4 | 22.1 |

To facilitate functional comparisons, Rz were also designed by picking sites predicted to be accessible for binding using the mFold program. Briefly, sites were chosen in mFold plots which had one flanking sequence predicted to lie within a single-stranded region, with the nucleotide triplet at a “transition”, and the other flanking sequence predicted to lie within a double-stranded region. These characteristics have consistently been observed for nearly all library-selected sites we have identified (currently encompassing more than 50 sites within 10 target RNAS).

The library-selected ribozymes (sRz) and those designed using computer models, based either on secondary structure (designated m1Rz; using mFold 2.3, (Zuker & Stiegler, 1981, Nucl. Acids Res. 9:133-148)) or single-stranded counts (designated m2Rz, using mFold 3.0, (Zuker & Jacobson, 1998, RNA 4:669-679)) were transcribed for in vitro cleavage test experiments.

The catalytic activities of sRz and mRz were determined using single turnover conditions. A trace amount of [³²P]-labeled target-RNA was incubated with 40 or 200 nM Rz in 5 mM MgCl₂, 20 mM Tris-HCl (pH 7.4) at 37° C. for 30 minutes, and the cleavage products were separated by denaturing PAGE. Three of the sRz showed “high” activity during a 30 minute cleavage reaction, cleaving between 39-44% of the target RNA using 40 nM Rz and 48-71% of the target RNA using 200 nM Rz. One of the sRz and one mRz showed “intermediate” activity; they cleaved 8-10% of target-RNA at 40 nM Rz or 10-15% at 200 nM. Another mRz was inactive. In additional experiments, when the Rz concentration was reduced to 1.6 nM, cleavage products with the 3 highly active sRz were still visible after PAGE. Overall, 92% of sRz showed efficient activity levels, with 54% being highly active and 38% intermediately active. In contrast, none of the mRz were highly active; 50% showed intermediate or low activities, and 50% were inactive (Table 2).

TABLE 2
Summary of Rz relative catalytic activities.

| | | | Activity | | | |
|---|---|---|---|---|---|---|
| | | | (high, | intermediate, | | inactive |
| Target | Rz, NUH | | ###; | ##; | low, #; | —) |
| HBV | sRz-885, | CUC | ### | | | |
| | sRz-469, | CUC | | ## | | |
| | sRz-408, | GUC | ### | | | |
| | sRz-777, | AUC | ### | | | |
| | m1Rz-247, | GUC | | ## | | |
| | m1Rz-355, | GUC | | | | |
| Pol I | sRz-458, | CUA | ### | | | |
| | sRz-353, | AUC | ### | | | |
| | sRz-595, | AUC | | ## | | |
| | sRz-70, | GUC | ### | | | |
| PTEN | sRz-281, | AUC | | ## | | |
| | sRz-681, | CUC | | ## | | |
| | sRz-425, | AUC | ### | | | |
| | sRz-499, | GUC | | ## | | |
| | sRz-774, | CUC | | | # | |
| | m1Rz-127, | CUU | | | | — |
| | m1Rz-151, | AUU | | ## | | |
| | m1Rz-439, | UUA | | | | — |
| | m1Rz-760, | AUC | | | | — |
| | m2Rz-227, | AUU | | ## | | |
| | m2Rz-304, | AUC | | | # | |
| | m2Rz-414, | AUA | | | # | |
| | m2Rz-961, | CUA | | | | — |

| | (high, | intermediate, | | inactive |
|---|---|---|---|---|
| | ###; | ##; | low, #; | —) |
| No. of sRz | 7 | 5 | 1 | |
| % | 53.8 | 38.5 | 7.7 | |
| No. of m1Rz | | 2 | | 4 |
| % | | 33.3 | | 66.7 |
| No. of m2Rz | | 1 | 2 | 1 |
| % | | 25 | 50 | 25 |

For kinetic analyses, 40 nM Rz and 1 to 100 nM of target RNA were incubated for various periods (ranging from 20 seconds to 120 minutes), to obtain kinetic data for both single and multiple turnover conditions. Results for the HBV-targeted sRz showed a Km of 26 nM, with a Kcat/Km of 1×106 (M⁻¹ min⁻¹). Similar analyses for the other sRz showed Kcat/Km values of 0.6×106 (M⁻¹ min⁻¹). In comparison, Kcat/Km values obtained for mRz to these and other targets (Benedict et al., 1998, Carcinogenesis 19:1223-1230, Ren et al., 1999, Gene Ther. Mol. Biol. 3:257-269, and Crone et al., 1999, Hepatology 29:1114-1123) are typically an order of magnitude lower.

To test the effectiveness of the sRz in cells, HepG2 cells (a human hepatoblastoma cell line), which can support HBV replication and secretion after transfection with HBV DNA (they cannot be directly infected with virus), were used. HepG2 cells were co-transfected with an HBV DNA construct and HBV-targeted sRz in the CLIP Triple ribozyme cassette (Benedict et al., 1998, Carcinogenesis 19:1223-1230, Ren et al., 1999, Gene Ther. Mol. Biol. 3:257-269, and Crone et al., 1999, Hepatology 29:1114-1123). The CLIP cassette encodes 2 cis-acting Rz flanking an internal, transacting Rz targeted to HBV. The 2 cis-acting Rz function to release themselves from the primary transcript, liberating the trans-acting internal hammerhead Rz with minimal non-specific flanking sequences, a process which affords significant advantages.

The HBV construct and the Trz constructs were co-transfected into HepG2 cells, and cultures were analyzed for the effects of sRz on HBV replication. At 4 and 5 days after transfection with the CLIP constructs containing sRz777 or sRz885, a dramatic inhibition of secretion of HBV was observed, and this was accompanied by inhibition of HbsAg secretion and by major reductions in HBV RNA target transcripts. The target sites for sRz777 and sRz885 are located in positions such that all 3 major HBV transcripts are targeted. For comparison, an mRz408 CLIP construct was also employed, which contained nucleotide substitutions in the 5′ flanking sequence; this Rz showed “intermediate activity” cleaving HBV target at approximately 20% of the rate at which sRz408 did, an activity which was equivalent to that of mRz247. The mRz408CLIP construct was not effective in blocking HBV replication. In addition, a CLIP construct targeted to an mFold-selected site showed no activity against HBV in this system.

Two additional repeat experiments with the 777 and 885 CLIP constructs also demonstrated marked reductions in secretion of HBV, although the reductions in HbsAg secretion and HBV RNA transcripts were more variable.

In summary, this library-selection procedure provides a relatively straightforward method for determining accessible sites in long target RNAs. Reamplification and transcription of selected guide RNA pools have been streamlined, and Rz targeted to the identified regions have been shown to be very active in vitro. In addition, the selected Rz targeted to HBV have also been shown to be efficacious in a cell culture model for HBV replication, suggesting the utility of the modified SELEX method in designing hammerhead Rz that are active in vivo.

Materials and Methods

Construction of an Antisense DNA Library and Target RNA Templates

A single stranded DNA library containing >10⁹ sequences (750 μg of DNA) was constructed by automated solid-state synthesis (Macromolecular Core Facility, Hershey, Pa.). The cleavage site was maintained by designating a central TC (that generates RNA with a central GA which is reverse complementary to UC of the target-RNA's triplet), while the sequence diversity was created by randomizing two domains (9 Ns and 6 Ns) flanking that TC, and fixing both 5′/3′-ends: The library sequence was 5′-GCCTCTAGAGTCGAANNNNNNNNNTCNNNNNNAGTGTTCTTCAGTCCC-3′. The 5′-end primer (P2, 5′-GCCTCTRAGAGTCGAA-3′, containing a Xba I restriction endonuclease site) and 3′-end primer (P3, 5′-CCGAAGCTTAATACGACTCACTATAGGGACTGAACACT-3′, containing a Hind III restriction endonuclease site and a T7 RNA polymerase promoter) were designed to utilize polymerase chain reaction (PCR) amplification of the randomized sequence in order to construct the double-stranded DNA library. The library was sequenced to confirm its composition. The library was then transcribed using T7 RNA polymerase to generate a random pool of multiple copies of approximately a billion different guide-RNA sequences.

Target RNA templates were produced by PCR for HBV and human Pol I. The HBV construct represented strain ayw, (GenBank Accession #V01460). The Pol I construct comprised nt 15-1053 of the hRPA39 subunit of human RNA polymerase I, (GenBank Accession #AF008442).

Reverse transcription/PCR (RT/PCR) was used to generate the PTEN construct. This was performed using total RNA isolated from C3H/10T1/2 cells (Clone 8, ATCC CCL-226 cell line). Total RNA was isolated using TRIzol Reagent (GibcoBRL), and the RT was performed using a modified method with the SuperScript™ II Rnase H-Reverse Transcriptase (GibcoBRL). 1 μg of total RNA, in a volume of 12 μL 20 mM Tris-HCl (pH 7.4), was heated with 10 pM of primer (P4, 5′ GACGAGAAGCTTTCAGACTTTTGTAATTTGTGTAGT-3′) at 85° C. for 3 minutes. The temperature was gradually decreased to 25° C. over 30 minutes, after which the other components were added according to manufacturer's instructions, and incubation was at 48° C. for 1 hour to generate cDNAs of PTEN.

PCR construction of double-stranded DNA for production of target RNA transcripts utilized Platinum Taq DNA Polymerase (GibcoBRL) and 5′-end primers containing either a T7 or Sp6 RNA polymerase promoter (T7 for Pol I and HBV, and Sp6 for PTEN). The 5′-primers used were: P5, 5′-CCGAAGCTTAATACGACTCACTATAGGGCATGTATTCAATCTAAGCAGGCT-3′ for HBV; P6, 5′-CCGAAGCTTAATACGACTCACTATAGGGGGCTTCTCAGGCGGTGGAGG-3′ for Pol I; and P7, 5′-CCGCACTATTTAGGTGACAGTATAGAAGCTTATGACAGCCATCATCAAAGAGAT-3′ for PTEN). The 3′-primers used were: P8, 5′-ACTGAAGGAAAGAAGTCAGAAGGC-3′ for HBV; P9, 5′-TCAGTCCATCTCAACTGCAT-3′ for Pol I; and the P4 primer utilized for the RT step described above for PTEN.

For comparative purposes, a number of mRz were transcribed using various primer pairs (Table 3). All constructs were sequenced in their entirety prior to use.

TABLE 3
Oligonucleotides for making selected/mFold Rzs.

| | | |
|---|---|---|
| | | 9N 6N |
| HBV | sRz-408 | 5′---TTCTCGGGG---GCTTGG---3′ |
| | sRz-469 | 5′---GGGCGCACC---TCTTTA---3′ |
| | sRz-777 | 5′---TCTGCCTAA---ATCTCT---3′ |
| | sRz-885 | 5′---TGGAGTTAC---TCGTTT---3′ |
| | m1Rz-247 | 5′---CGCAGCAGG---TGGAGC---3′ |
| | m1Rz-355 | 5′---CGCGGGACG---CTTTGT---3′ |
| Pol I | sRz-70 | 5′---TCGCAATGT---CATACT---3′ |
| | sRz-353 | 5′---TCATGCTGA---CCCGTC---3′ |
| | sRz-458 | 5′---CCATGCTGC---AAAGAT---3′ |
| | sRz-595 | 5′---ATATCCTCA---GCTCAG---3′ |
| PTEN | sRz-281 | 5′---TGAAGACCA---ACCCAC---3′ |
| | sRz-425 | 5′---TTTATTGCA---GGGGCA---3′ |
| | sRz-499 | 5′---AAAAGGGAG---ACAATTT---3′ |
| | sRz-681 | 5′---ATATATTCC---CAATTC---3′ |
| | sRz-774 | 5′---GTAGAGTTC---CCACA---3′ |
| | m1Rz-127 | 5′---CAGAAAGAC---GAAGGT---3′ |
| | m1Rz-151 | 5′---GGAACAATA---GATGAT---3′ |
| | m1Rz-439 | 5′---GCAAATTTT---AAGGCA---3′ |
| | m1Rz-760 | 5′---GTGGTGATA---AAAGTA---3′ |
| | m2Rz-227 | 5′---TGAGAGACA---ATAACA---3′ |
| | m2Rz-304 | 5′---TAGAACTTA---AAACCC---3′ |
| | m2Rz-414 | 5′---ATTTGTGCA---TTTATT---3′ |
| | m2Rz-961 | 5′---TACTCACCC---ACAAAA---3′ |

The oligonucleotides used for making the library-selected and mFold-designed Rz are shown in the table. They include:
(1) A 5′-end fixed sequence of 5′-GACCCTTGGAATTC-3′;
(2) A central catalytic core sequence of 5′-TTTCGTCCTCACGGACTCATCAG-3′; and
(3) A 3′-end fixed sequence of 5′-GGATCCTGGAACCCTATAG-3′.
Designations for the Rz were based upon locations of the cutting sites within the transcripts used for selection or mFold plots.

Transcription of Library Guide-RNA and Target-RNA

To generate the guide-RNA library, sets of oligonucleotides were synthesized (GibcoBRL), which consisted of a central catalytic core domain of the hammerhead Rz (23 nt), flanked by two variable domains (9 Ns adjacent to the 5′-end and 6 Ns adjacent to the 3′-end, to pair with the target-RNA), and fixed 5′/3′-end sequences: P9, 5′-GACCCTTGGAATTC-9N-TTTCGTCCTCACGGACTCATCAG-6N-GGATCCTGGAACCCTATAG-3′ (Table 3). The double stranded DNA templates for in vitro transcription were made by single-cycle PCR with a 3′-end primer (P10, 5′-CCGAAGCTTAATACGACTCACTATAGGGTTCCAGGATCC-3′) containing a T7 RNA polymerase promoter.

Both the library guide-RNA pool and target-RNA were transcribed in vitro using the Riboprobe System (Promega) with [³²P]-CTP; T7 or Sp6 RNA polymerases were utilized and reactions were performed at 37° C. for 2 hours, followed by a RNase-free DNase digestion to destroy the template DNAs. The transcripts were extracted with phenol/chloroform, heated at 85° C. for 3 minutes in an equal volume of loading buffer (80% formamide, 100 mM EDTA, pH 8.0, 0.05% bromophenol blue, 0.05% xylene cyanol FF) and purified by PAGE (Benedict et al., 1998, Carcinogenesis 19:1223-1230). The corresponding bands were excised, homogenized in buffer (20 mM Tris-HCl, pH 7.4, 250 mM NaCl) and then incubated for 2 hours at 4° C. and then for 5 minutes at 85° C. Following centrifugation at 2000×g for 5 minutes the supernatant was removed, and the RNA was precipitated with ethanol and resuspended in 20 mM Tris-HCl (pH 7.4).

In vitro Guide-RNA Library Selection

At least five rounds of selection were performed for each target RNA. Each round of selection was performed as follows: 10 micromoles Guide-RNA pool and 0.1 micromoles target-RNA were diluted with 20 mM Tris-HCl (pH 7.4) in separate tubes, heated to 56° C. for 5 minutes and then cooled to 37° C. 5 mM MgCl₂ was added to each of the tubes and they were incubated for an additional 5 minutes at 37° C. The contents of both tubes were mixed together gently (total 20 μL) and incubated for 15 minutes, allowing RNA-RNA complexes to form, after which ⅕ volume of loading buffer (20% glycerol plus 0.05% bromophenol blue and 0.05% xylene cyanol FF) was added. The bound complexes were separated from the unbound guide-RNA pool in a 8% urea-free polyacrylamide-TBE gel. The RNA-RNA complexes (containing the bound species from the guide RNA library) were isolated and purified as described above, and resuspended in 20 mM Tris-HCl (pH 7.4). The selected guide-RNAs were reverse transcribed to produce their cDNAs using primer P2 (as described above), subjected to PCR-amplification using primers P2 and P3, and subsequently transcribed using T7 polymerase to produce a new guide-RNA pool which was enriched for better target-RNA-binding sequences for each specific target-RNA.

Each of these new guide-RNA pools was again selected using the corresponding target-RNA to begin the next round. The selection stringency was increased by reducing (by half) the target-RNA concentration as the number of selection rounds increased. After 5 rounds (6 rounds for HBV), the selected pools of guide-RNAs were tested for the ability to bind the corresponding target-RNA respectively.

The [³²P]-labeled guide-RNA pool obtained after 5 rounds of selection was incubated (at 1 nM) with various concentrations of unlabeled target-RNA under conditions described above in the previous paragraphs (and also in Pan et al. 2001), and the samples were then analyzed by PAGE using an 8% urea-free gel. The gel was dried, then exposed to autoradiographic film and quantitated using a Phosphor-Imager (Molecular Dynamics).

The PCR products of 5th round selection were cloned into pCR2.1-TOPO directly, or were cloned into pCRII using Hind III and Xba I restriction endonucleases (TOPO TA Cloning Kit, Invitrogen). About 30 clones from each selected guide-RNA pool were sequenced (reagents were from USB, using Sequenase T7 DNA polymerase and 7-deaza-dGTP), and aligned to the corresponding target-RNA with the MacVector™ 5.0 program. For comparative purposes, a set of cutting sites was also chosen using secondary structural or single-stranded frequency predictions using mFold-modeling of target-RNAs (Zuker & Jacobson, 1998, RNA 4:669-679 and Zuker & Stigler, 1981, Nucleic Acids Res 9:133-148).

In Vitro Cleavage Tests

Rz targeted to the individual library-selected sites were transcribed from double-stranded DNA oligonucleotides (Table 3) using T7 (HBV and Pol I) or Sp6 (PTEN) polymerase as described for generation of the guide-RNA library. For standard screening of Rz activity, incubations contained trace amounts of [³²P]-labeled target RNA, 40 nM Rz RNA, and were for 30 minutes (or 2 hours) at 37° C. in 20 mM Tris-HCl (pH 7.4), 5 mM MgCl₂. After the conclusion of the incubations, samples were separated in a urea-polyacrylamide gel; the gels were then dried and radioactivity was analyzed using a Phosphor-Imager.

For kinetic analyses, a trace amount of [³²P]-labeled target-RNA was mixed with unlabeled target-RNA (to yield final concentrations of 1, 10 or 100 nM target RNA) and Rz-RNA (40 nM final concentration) and incubations were performed using the same conditions as for the in vitro library selection described above, except that incubation times were varied (for 20 seconds, 40 seconds, 1 minute, 3 minutes, 10 minutes, 30 minutes and 2 hours). The samples were then separated in a urea-polyacrylamide gel, and then dried and analyzed using a Phosphor-Imager.

Effects of Rz on HBV Replication in Cell Culture

To test the effectiveness of sRZ in cell culture, HepG2 cells were maintained in minimal essential medium supplemented with 10% heat-inactivated fetal bovine serum, in a humidified incubator at 30° C. with 5% CO₂. These cells were co-transfected with pBB4.5HBV1.3 (a 1.3× unit length HBV DNA plasmid construct; see Delaney & Isom, 1998, Hepatology 28:1134-2246) and either pLSCLIP, pLSCLIPmRz408, pLSCLIPsRz777, or pLSCLIPsRz885 (PLSCLIP denotes the CLIP cassette in the LacSwitch vector, from Stratagene). pLSCLIPsRz777 and pLSCLIPsRz885 were constructed by annealing reverse complementary oligonucleotides (CLAW437/CLAW438 and CLAW397/CLAW398, respectively) and then inserting them into the Bgl II site of pLSCLIP. pLSCLIPmRz408 was constructed the same way with oligonucleotides CLAW435/CLAW436. However, these oligonucleotides were inadvertently synthesized so that the 5′ flanking region contained mismatches; subsequent testing in vitro showed that this Rz had approximately 20% of the catalytic activity of the sRz408, which was equivalent to the activity of mRz247, and it was therefore included in the experiments as an “intermediate” comparison.

HepG2 cells were transfected using FuGENE6 transfection reagent (Boehringer Mannheim). A total of 5 μg of DNA (0.5 μg pBB4.5HBV1.3 and 2.7 μg of the PLSCLIP constructs), 24 μL of enhancer, and 30 μL of Effectene transfection reagent. The cells were incubated in the DNA/reagent mixture in serum-containing medium for 6 hours.

For Northern blot analyses, total RNA was isolated from transfected HepG2 cells four and five days post-transfection (Chomczynski & Sacchi, 1987, Anal Biochem 162:156-159), and Northern Blot analysis was performed using 10 μg of total RNA as described (Davis et al., 1986, Preparation and analysis of RNA from eukaryotic cells. In: Basic Methods in Molecular Biology, New York: Elsevier Science Publishing Co., Inc., 129-156). Hybridization was performed using a [³²P]-radiolabeled HBV probe generated by random priming (with Boehringer Mannheim Random Prime DNA Labeling ktis). The blots were probed simultaneously for HBV and GAPDH transcripts. Following hybridizations, the blots were rinsed under high-stringency conditions and exposed for audoradiography.

For analysis of secreted extracellular HBV DNA, medium was collected on day 4 and day 5 post-transfection, and centrifuged at 6,000×g for 5 minutes to remove cellular debris. Triplicate samples were pooled and HBV particles were precipitated and analyzed as described in Wei et al., 1996 J. Virol. 70:6455-6458. Viral pellets were resuspended in PBS and digested with Proteinase K, then extracted with phenol/chloroform. DNA was precipitated with 0.1 volume of 3 M sodium acetate and 1 volume of isopropanol. Ten micrograms of tRNA was added as a carrier during precipitation. Pellets were resuspended in TE and digested with 0.5 mg/ml RNase for 1 hour. DNA was then analyzed by electrophoresis and Southern blotting, followed by autoradiography.

For analysis of secreted HBV Surface Antigen (HbsAg), detection was performed by radioimmunoassay using a Sorin Diagnostics kit. Medium from transfected cells was collected and centrifuged at 6,000×g to remove cellular debris. Total counts were compared for analysis.

### Example 2

### Ribozyme Library Screening

Library Construction

To generate the library, sets of oligonucleotides were synthesized, which consisted of a central catalytic core domain of the hammerhead Rz (23 nt), flanked by two variable domains (9 Ns adjacent to the 5′-end and 9Ns adjacent to the 3′-end, to pair with the target-RNA), and fixed 5′/3′-end sequences: 5′-CGC AGA CCC TTG GAA TTC NNN NNN NNN TTT CGT CCT CAC GGA CTC ATC AGN NNN NNN NNG GAT CCT GGA ACC GAC GAT-3′. The double stranded DNA templates for in vitro transcription were made by single-cycle PCR with a 5′ end primer (5′-3′-end primer (5′-GCC AAG CTA TTT AGG TGA CAC TAT AGA TCG TCG GTT CCA GGA TCC-3′) containing an Sp6 RNA polymerase promoter.

The RNAs were transcribed in vitro using the Riboprobe System (Promega) with [³²P]-CTP. Sp6 RNA polymerase was utilized and reactions were performed at 37° C. for 2 hours, followed by a RNase-free DNase digestion to destroy the template DNAs. The transcripts were extracted with phenol/chloroform, heated at 85° C. for 3 minutes in an equal volume of loading buffer (80% formamide, 100 mM EDTA, pH 8.0, 0.05% bromophenol blue, 0.05% xylene cyanol FF) and purified by PAGE (Benedict et al., 1998, Carcinogenesis 19:1223-1230). The corresponding bands were excised, homogenized in buffer (20 mM Tris-HCl, pH 7.4, 250 mM NaCl) and then incubated for 2 hours at 4° C. and then for 5 minutes at 85° C. Following centrifugation at 2000×g for 5 minutes the supematant was removed, and the RNA was precipitated with ethanol and resuspended in 20 mM Tris-HCl (pH 7.4).

Random Selection of Ribozymes

100 pM library RNA (approximately 1000 copies for each sequence), and 1 pM target RNA was used as the starting material. The RNA mixture was heated in 100 μL of 20 mM Tris-HCl (pH 7.5) at 85° C. for 3 minutes, cooled down at room temperature for 15 minutes, then chilled on ice for 5 minutes. 20 μL of 6× DNA loading buffer (20% glycerol with dyes) was added and the resultant mixture was electrophoresed on an 8% “native” polyacrylamide gel to isolate the library RNA species which bouiid to the targeted RNA. The isolated library RNA was used as substrate for a reverse transcription reaction, using Omniscript reverse transcriptase (Qiagen) and standard conditions. PCR amplification of the RT product was performed to produce the selected DNA species. This constituted a “round” of selection. The above steps were repeated once.

Library Screening and Mapping

100 pM selected library RNA were mixed with 0.1 pM 5′/3′ 32P-end-labeled target RNA. Each of the RNA mixtures were incubated separately at 65° C. for 3 min in 20 mM Tris-HCl, pH 7.5, and then at 37° C. for 3 min. MgCl₂ was added to a final concentration of 50 mM, and heat incubated at 37° C. for 3 min. The 2 samples were mixed thoroughly, and incubated at 37° C. for 2 h (total volume of 5 μL). 1 μL of 0.5 M EDTA (pH 8.0) and 6 μL of 10 M urea with dyes were added to the mixture.

A “G-ladder” and a “base hydrolysis ladder” sample were prepared for PAGE. 0.1 pM 5′/3′ ³²P-end-labeled target RNA was suspended in 12 μL 5 M urea, 15 mM NaCitrate (pH 3.5), 1 mM EDTA, 1.5 μg tRNA (E. coli), with dyes, 0.2 Units RNase T and incubated at 50° C. for 15 minutes. This enzymatic digestion cleaves after G residues. 0.1 pM 5′/3′ ³²P-end-labled target RNA was suspended in 6 μL of 50 mM NaHCO₃/NaCO₃ (pH 9.0), 1 mM EDTA, 1.5 μg tRNA (E. coli). The mixture was boiled for 8 minutes, then 6 μL of 10 M urea with dyes was added.

The samples were then separated by PAGE in a standard 8% sequencing gel. The sequences of the major cut sites were identified by comparison with the G- and base hydrolysis ladders. This procedure followed that of Donis-Heller, 1980, Nucleic Acids Research 8:3133-3142.

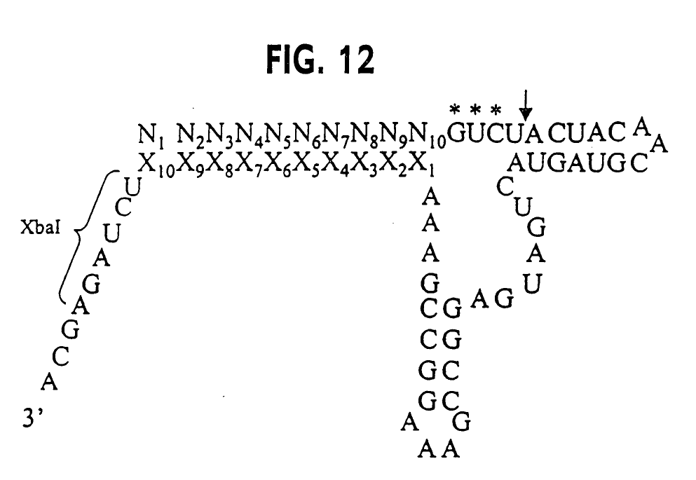

The above procedure was modified when the target RNA was to be ³²P-labeled at the 3′ end. A cis-acting hammerhead ribozyme sequence was added to the target RNA. Its action produced a precise 3′-end. This allowed identification of sites closer to the 3′ end, since otherwise microheterogeneity at the 3′ end precluded direct 3′ end labeling. Prior to end-labeling, the cyclic phosphate bond of the 3′-terminal C was broken by incubating the RNA in 10 mM HCl at 25° C. for 4 hours. The RNA was then labeled with ³²P-CoTP using poly(A) polymerase. The basic format was to add a catalytic hammerhead ribozyme domain and a 3′ flanking sequence of 10 nt which was reverse complementary to the 3′ end of the particular target RNA undergoing library selection (FIG. 12).

The protocol for screening a riboyzme library was essentially similar to the protocol described herein except for the identification of cleavage sites. Instead of cloning the selected guide-RNA pool into vectors for sequencing, the selected ribozyme sequences may be identified by the cut products.

### Example 3

### DNAzyme Library Screening

DNAzyme Library (Dz) Construction

The RNA target has a sequence of 8 random nucleotides flanking (A/G)-(C/T), followed by 7 random nucleotides, i.e., 5′-N8-(A/G)-(C/T)-N7-3′. The DNA library has a BS14 primer upstream of 7 random nucleotides flanking a 15 nt catalytic core, followed by 8 random nucleotides and a TS15 primer, i.e., 5′-GAC CCT TGG AAT TCN-N7-RGG CTA GCT ACA ACG A-N8-CTA ATT AAG CTT CGG-3′.

Library “Pre-Selection”

The target RNA and the Dz library are heated together in the absence of Mg²⁺ at a temperature sufficient to denature the secondary structure of the nucleic acids and cooled to room temperature, and bound RNA-DNA complexes are isolated on a nondenaturing gel. PCR is performed using the BS14 and TS15 primers to reamplify the pre-selected library species. Multiple rounds of PCR are then run using only primer BS14, to amplify the pre-selected library in a unidirectional fashion. The PCR steps may be repeated if necessary

Selected Dz Library Screening

Screening is conducted as described for the Rz Library screening. Briefly, 5′ and/or 3′ ³²P-labeled target RNA is incubated with the preselected Dz library; “G” and base hydrolysis ladders are generated using the same ³²P-labeled target RNA preparations; and the results are analyzed on a sequencing gel. The major cleavage products are defined by comparison with the G and base-hydrolysis ladders on the gel, and Dz are then designed based on comparison with the target RNA sequence in the identified regions.

### Example 4

### Effects of Antisense Oligonucleotides Targeted to a Library Selected Site in Transgenic Mice

As shown in the data presented in Tables 4 to 7, a DNAzyme or its catalytically inactive counterpart (i.e., an antisense oligonucleotide), was effective in reducing HBV secretion in a transgenic mouse that expresses human HBV, i.e., in vivo.

TABLE 4
Effects of DNAzyme (Dz879) or the catalytically inactive counterpart
(m879) on serum HBV genome equivalents in HBV Transgenic Mice.
50 μg of Dz879 or m879 were administered in asialofetuin-coated
liposomes twice per week for 2 weeks, and sacrificed 48 h after the
final treatment. As is evident, both Dz879 and m879 were effective
in reducing HBV secretion in vivo after 2 weeks of treatment.
However, the effect was diminished after 5 weeks, presumably
because of an immune response to the asialofetuin.
Serum HBV Genome Equivalents/ml (× 10⁻³)

| Group 1 | | | | |
|---|---|---|---|---|
| Dz, 2 | Group 3 | Group 5 | Group 7 | Group 9 |
| weeks | Dz. 5 weeks | mDz, 2 weeks | mDz, 5 week | Control |
| 0.4 ± .38 | 3.29 ± 3.35 | 0.74 ± 0.85 | 4.10 ± 3.19 | 6.57 ± 3.31 |
| P values, | | | | |
| Student's | | | | |
| t-test | | | | |
| 0.0025 | 0.072 | 0.003 | 0.180 | |

TABLE 5
Effects of Dz879 and m879 on HBV Core Ag in liver of HBV
Transgenic Mice. Dz879 and m879 were administered in asialofetuin-
coated liposomes as described. Liver tissue was obtained, fixed,
processed, and immunohistochemistry was performed for HBV Core
antigen around central veins. As is evident, there is a dramatic
reduction in staining for HBV Core antigen after 2 weeks.
In addition, the intensity of staining was also greatly reduced,
indicating an even more marked effect than is shown by the
cytoplasmic staining numbers.

Animals: Female Transgenic mice (founder 1.3.32)
Treatment schedule: twice per week, (Tue, Friday) × 2 or 5 weeks
Virus: Human hepatitis B virus
Treatment route: i.p.
Drug: Prepared at Penn State sent to USU
Experiment duration: 2 or 5 weeks

| | Mean HbcAg-stained cytoplasms/total | |
|---|---|---|
| | cells^{a} ± standard devation (n^{b}) | |
| Treatment | Day 14^{c} | Day 35 |
| CL-ASF-DNAzyme | 0.04 ± 0.05 (10)*** | 0.14 ± 0.12 (10)*** |
| CL-ASF-DNAzyme mutant | 0.15 ± 0.13 (10)*** | 0.30 ± 0.15 (9)* |
| No Treatment | 0.47 ± 0.18 (10) | |

^{a}Number of stained cytoplasms per total number of cells around lumen of central veins. Average of 5 veins counted.
^{b}Number of animals in each treatment group.
^{c}Days after initial treatment.
*P < 0.05, compared to no treatment.
***P < 0.001, compared to no treatment.

TABLE 6
Effects of Dz879 and m879 on HBV RNA Transcripts in HBV
Transgenic Mice. Dz879 and m879 were administered as described,
and liver tissue was extracted for RNA. RNA was analyzed by
Northern blot analysis followed by densitometry; results showed
major reductions in HBV RNA transcript levels (all transcripts, as
was the case with cell culture results).

Animals: Female Transgenic mice (founder 1.3.32)
Treatment schedule: twice per week, (Tue, Friday) 2 weeks
Virus: Human hepatitis B virus
Treatment route: i.p.
Drug: Prepared at Penn State sent to USU
Experiment duration: 2 or 5 weeks

| Treatment | Relative Liver HBV RNA^{a} ± SD (n^{b}) |
|---|---|
| CL-ASF-DNAzyme | 7.0 ± 3.5 (10)* |
| CL-ASF-DNAzyme mutant | 9.4 ± 5.7 (10)* |
| No Treatment | 16.0 ± 5.6 (10) |

^{a}Mean signal of HBV RNA normalized to GAPDH housekeeping gene using Northern blot analysis ± standard deviation.
^{b}Number of animals in each treatment group.
*P < 0.0001, compared to no treatment group.
Note:
The RNA samples from 5 week-treatment were degraded.

TABLE 7
Effects of Dz879 and m879 on HBV liver DNA in Transgenic Mice.
Dz879 and m879 were administered in asialofetuin-coated liposomes
as described (legend to Table III). Liver tissue was extracted for
DNA, and HBV genomic DNA was quantitated by cross-over PCR.
Administration of Dz879 and m879 resulted in a dramatic reduction
in HBV liver DNA.

Animals: Female Transgenic mice (founder 1.3.32)
Treatment schedule: twice per week, (Tue, Friday) × 2 or 5 weeks
Virus: Human hepatitis B virus
Treatment route: i.p.
Drug: Prepared at Penn State sent to USU
Experiment duration: 2 or 5 weeks

| | Liver HBV DNA | |
|---|---|---|
| | Mean log₁₀ fg/ug cel DNA ± sd (n^{a}) | |
| Treatment | Day 14^{b} | Day 35 |
| CL-ASF-DNAzyme | 1.9 ± 0.22 (10)* | NT^{c} |
| CL-ASF-DNAzyme mutant | 1.75 ± 0.21 (10)* | NT^{c} |
| No Treatment | 4.11 ± 0.34 (8) | |

^{a}Number of animals in each treatment group.
^{b}Days after initial treatment.
^{c}Faulty preparation of samples, invalid results.
*P < 0.01, compared to no treatment.

### Example 5

### Rapid Screening of Efficient Target Cleavage Sites Using a Hammerhead Ribozyme Library

In this example, we have constructed a Rz library with randomized annealing arms and fixed 5′/3′-end flanking sequences (FIG. 1). Library selection with any transcript can be performed under magnesium-free annealing conditions, based upon Watson-Crick base-pairing. The selected Rz pool is reamplified by reverse transcription and PCR, and then used to cleave the target RNA in the presence of magnesium. Using full-length transcripts of human papillomavirus (HPV16-E6/E7, 782 nt in length; HPV11-E6/E7, 731 nt), human immunodeficiency virus (HIV-TAT, 264 nt), human malignant melanoma metastasis-suppressor (Kiss-1, 441 nt), human apoptosis inhibitor 4(API4, 557 nt), and mouse C9 subunit of the multicatalytic proteinase (MCP-C9, 1166 nt), we demonstrate that very active Rz can be rapidly and precisely engineered by analyzing the cleavage products of target RNAs produced with a pool of Rz-library RNA systematically isolated from the random Rz-library. Essentially all Rzs targeted to these library-selected sites cleaved their transcripts in vitro efficiently. In cell culture, all Rz targeted to HPV16 E6/E7 effectively reduced E6/E7 transcripts within cells, and a number of them also inhibited growth of SiHa cells.

Results and Discussion

In Vitro Selection of a Rz-Library by Annealing with Target RNAs

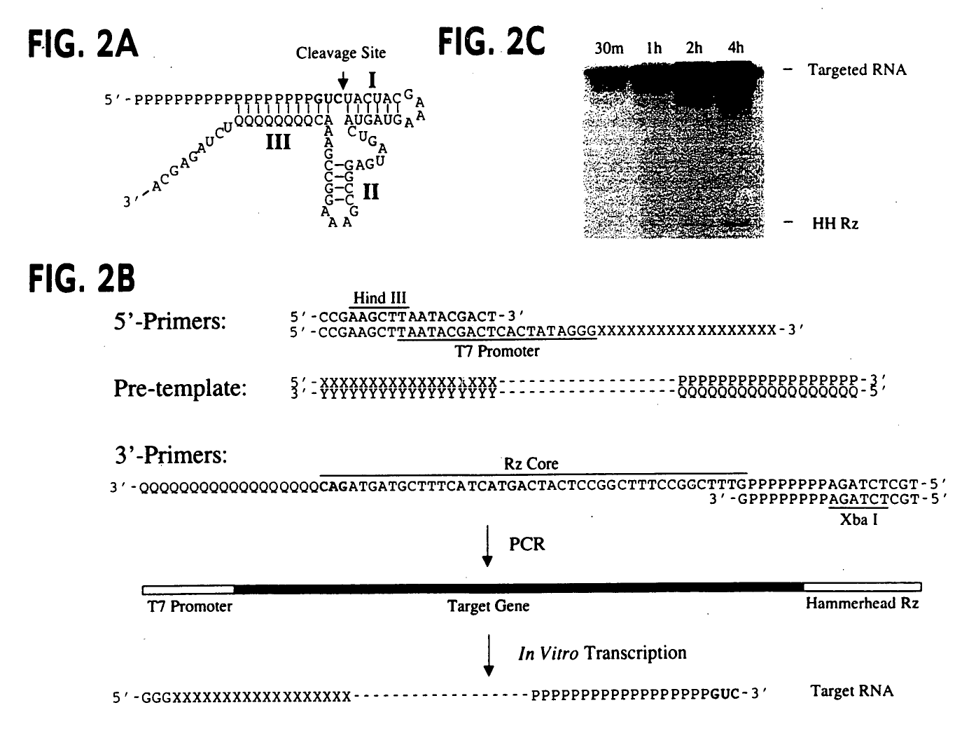

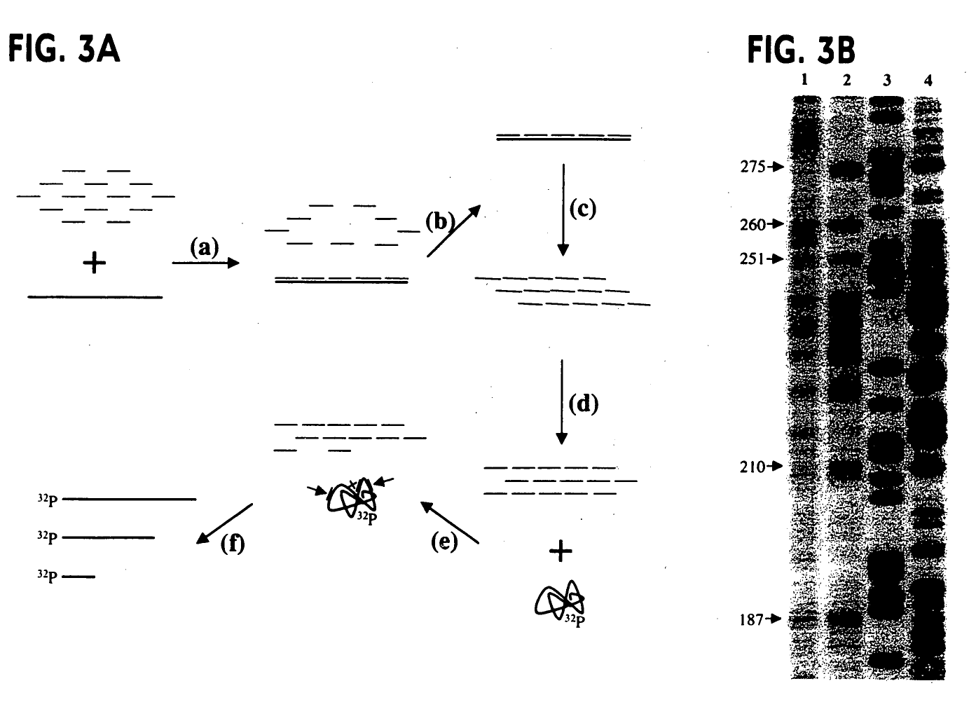

A double-stranded DNA library was used to generate a Rz-library with multiple copies of approximately 10¹⁰ different RNA sequences. Each transcript was 79 nt in length, with a central catalytic core flanked on each side by random sequences of 9Ns and by defined 5′/3′-end sequences (FIG. 1). DNA templates of targeted RNA were generated by PCR or RT/PCR with a T7 promoter in the 5′-primers. To circumvent the problem of microheterogeneity of transcripts at their 3′-ends, we used a 3′-primer encoding a self-cleaving Rz, so that transcripts with precise 3′-GUC ends were produced during in vitro runoff transcription (FIG. 2). The Rz library was subjected to selection with each of 6 different target-RNAs (HPV16, HPV11, HIV-TAT, Kiss-1, API4 and MCP-C9) under magnesium-free conditions, to allow isolation of RNA molecules that annealed to the corresponding target-RNA (FIG. 3A, (a) and (b)). The isolated annealed Rz-library RNA pool was subsequently amplified (FIG. 3A, (c)) by RT/PCR, and then subjected to a second round of selection at a lower target-RNA concentration, to increase the selection stringency, and to decrease background.

The re-amplified second round selected Rz (sRz) library RNA pools were used to cleave 5′ or 3′-end labeled target-RNA (FIG. 3A, (d) and (e)). The cleaved products were analyzed on sequencing gels, in comparison with G-, A-, or base-hydrolysis products (FIG. 3A, (f)). The cleavage sites were precisely identified on the corresponding target-RNA (FIG. 3B). 3-12 cleavage sites (11 for HPV16, 12 for HPV11, 6 for for HIV-TAT, 2 for Kiss-1, 3 for API4 and MCP-C9) were located on the corresponding target RNA. In addition, the intensity of the cleaved products reflected the catalytic activity of that sRz in a direct manner. The numbers of efficient cleavage sites were different among the target RNAs, presumably due to sequence specificity and folding structure.

In vitro Cleavage Kinetics of sRz

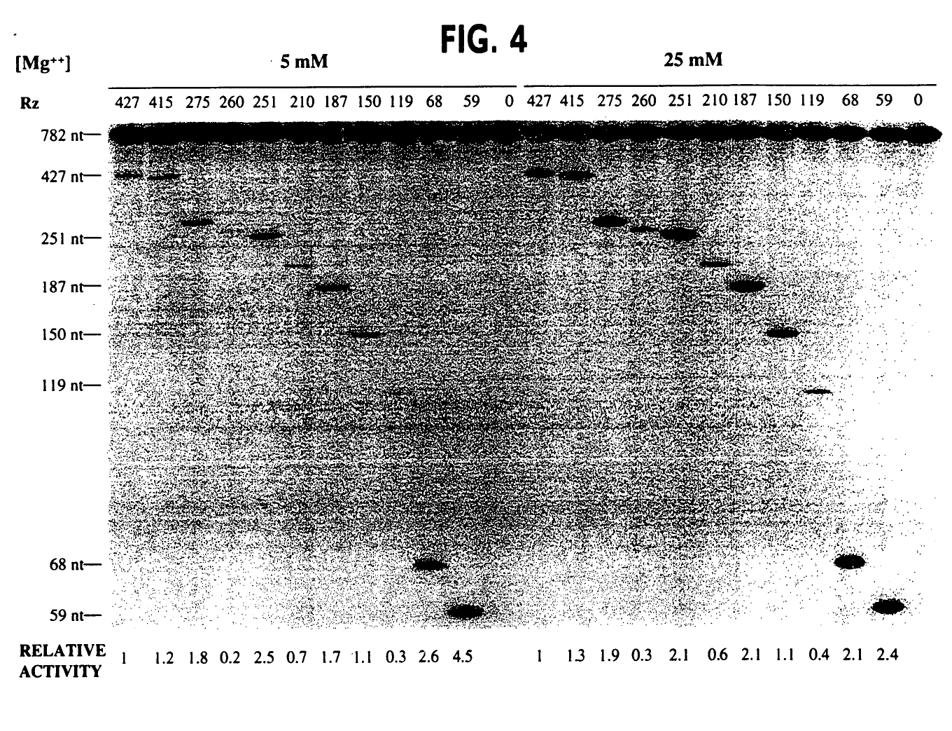

The catalytic activities of sRzs were determined using single turnover conditions. A trace amount of ³²P-labeled target-RNA was incubated with 40 nM Rz in buffer containing 20 mM Tris-HCl (pH 7.4) and varying concentrations of MgCl₂ (1, 5, or 25 mM) at 37° C. for 30 min. 25 mM MgCl₂ was used because it has been reported to yield cleavage rates similar to those observed in the presence of cytosol (Nedbal & Sczakiel 1997). Cleavage products were then analyzed by denaturing PAGE (see FIG. 4-6 for in vitro cleavage results for sRz targeted to HPV16). Seven (of 10) of the sRz showed higher activity than Rz427, the most active Rz previously selected using our oligonucleotide library procedure (Pan et al. 2001), with 2 others showing roughly comparable activity. Overall, fully 100% of sRz targeted to the identified sites have high efficiency in vitro, as previously defined (Pan et al., 2001; which is hereby incorporated by reference in its entirety), as compared with 40% identified using our modified SELEX oligonucleotide selection procedure.

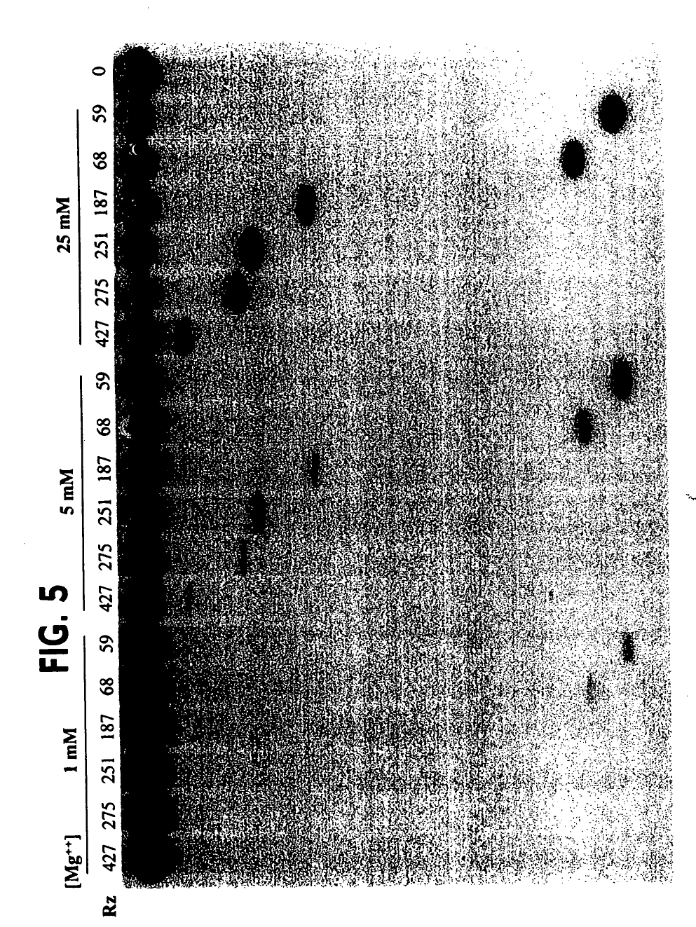

The higher concentrations of magnesium increased sRz catalytic activities, but decreased somewhat the differential between them. Compared with Rz427, the best selected sRz (Rz59) was 2.4 times more active at 25 mM of MgCl₂ and 4.5 times active at 5 mM MgCl₂ (FIG. 4), and only sRz59 and sRz68 showed demonstrable activity with 1 mM MgCl₂ (FIG. 5).

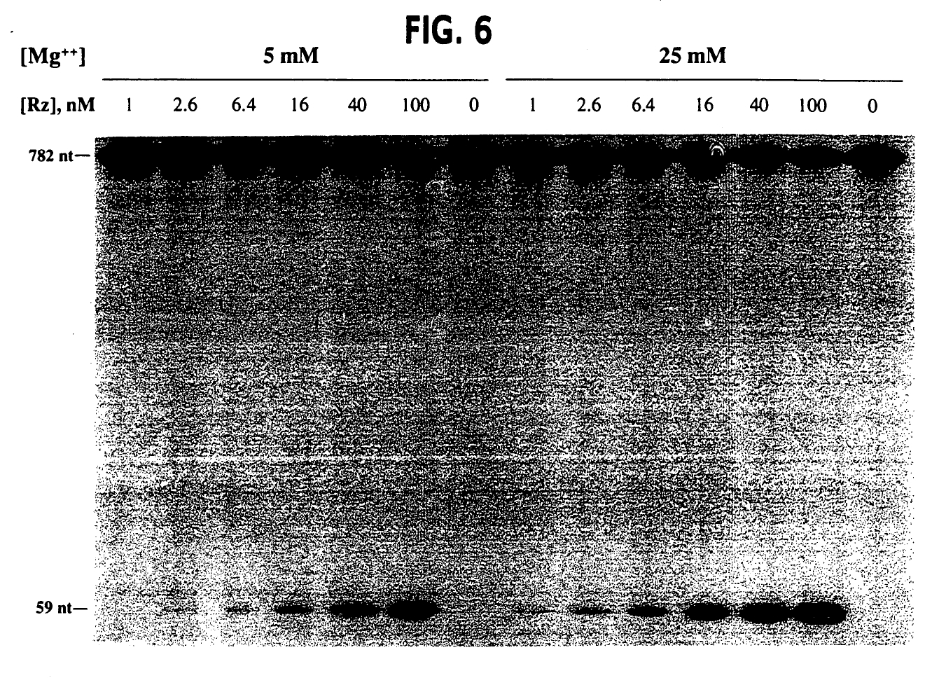

In additional experiments, when the Rz concentration was reduced to 1 nM in reactions containing 10 nM of target, the cleavage products with the sRz59 were still visible after PAGE and autoradiography (FIG. 6). To our knowledge, this is the lowest concentration of Rz shown to be effective versus a long target RNA. All of the sRz showed highly efficient cleavage of their full-length target RNAs, as previously defined (Pan et al., 2001). Analysis of the efficient cleavage sites for all of the six target RNAs (Tables 9 and 10) showed a tendency for a GUC-triplet, similar to that for other Rz, with GUU and GUA showing slightly lower prevalence. A distinguishing feature was two D nucleotides (A, G or U, but C) surrounding the triplet, yielding a consensus of 5′-DGUHD-3′.

For kinetic analyses, 100 nM Rz and 10-1000 nM of target RNA were incubated for various periods (ranging from 40 sec to 60 min), to obtain kinetic data for both single and multiple turnover conditions. Results for the HPV16-E6/E7 targeted sRzs generally showed Km's of 20-50 nM. sRz59 showed a Kcat/Km of 1.91×10⁶ (M⁻¹ min⁻¹), a value about 5 times higher than Rz427's Kcat/Km value of 0.34×10⁶ (M⁻¹ min⁻¹); this presumably reflects a faster chemical step of sRz59's catalytic activity, since Km values appear to be similar.

Effectiveness of sRz in Cell Culture

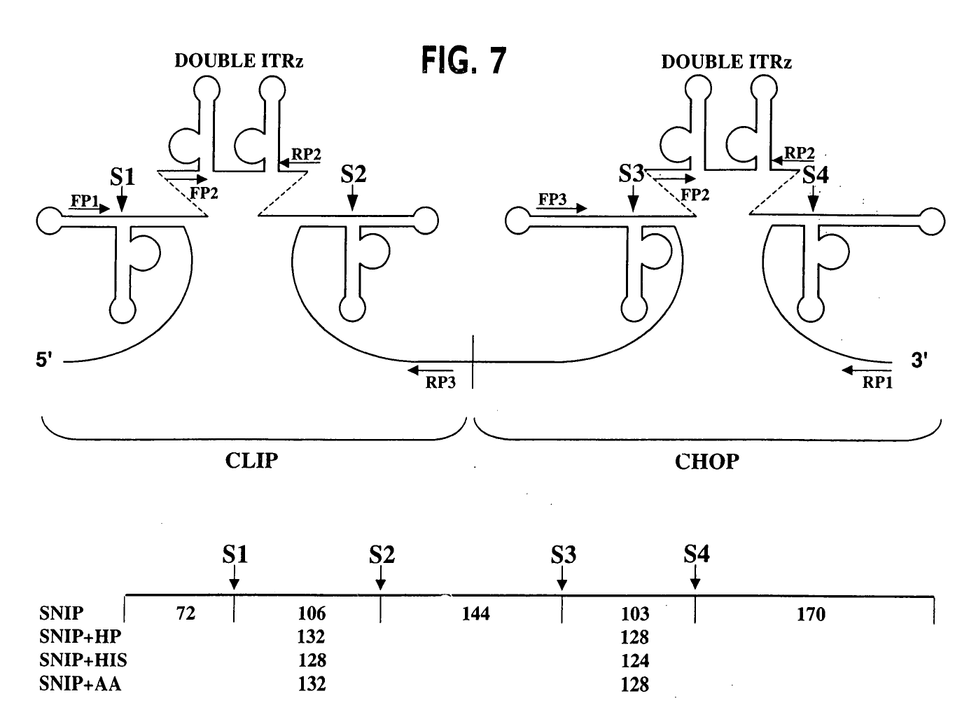

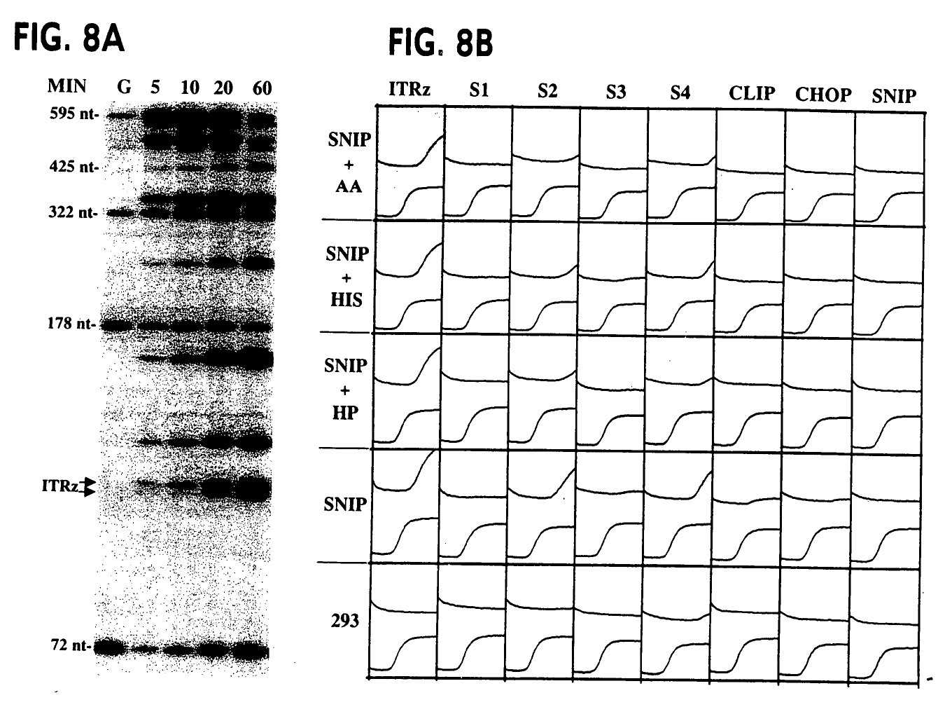

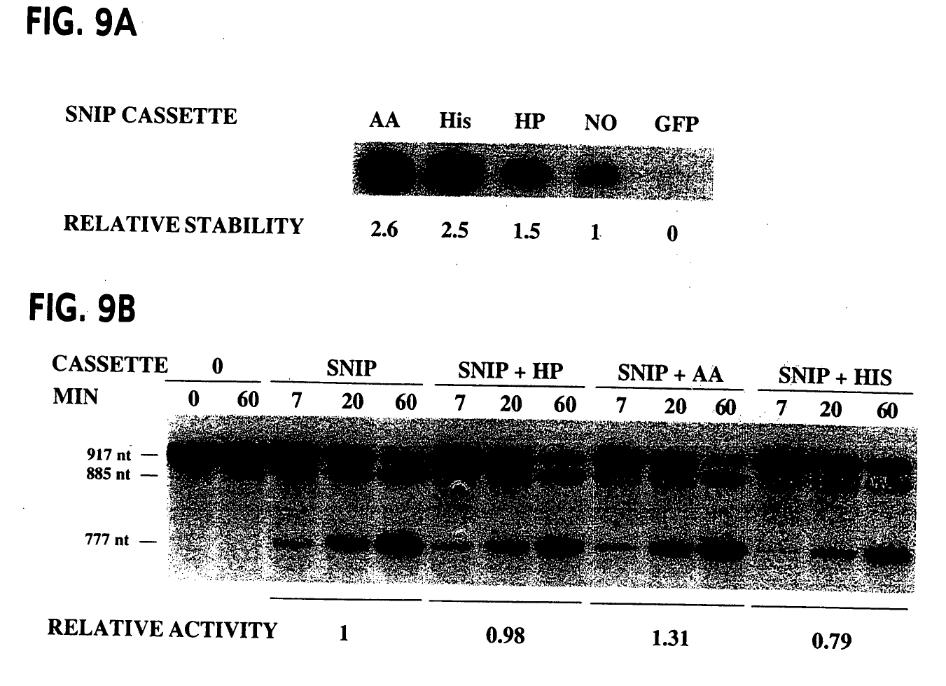

To test the effectiveness of the sRz within cells, we used 293T cells (a human embryonal kidney cell line, ATCC CRL-1573), which can be efficiently transfected with plasmid DNA (80-100% transfection efficiency was observed using a green fluorescent protein reporter construct. The sRz were placed within our SNIPAA cassette (FIG. 7). Briefly, this cassette contains 2 triple-Rz (TRz) cassettes, CLIP (Benedict et al 1998, Crone et al 1999, Ren et al 1998) and CHOP, each of which encodes 2 cis-acting Rz flanking an internal, trans-acting Rz targeted to the chosen RNA. The 2 cis-acting Rz function to release themselves from the primary transcript, liberating the trans-acting internal hammerhead Rz (ITRz) with minimal non-specific flanking sequences (FIG. 8). Two contiguous trans-acting Rz were found to slightly augment each other's catalytic activities, so that the liberated entity is referred to as a double internal trans-acting Rz (dITRz). Use of this cassette affords significant advantages, including enhanced activity of the liberated ITRz/dITRz, as well as a distribution of ITRz/dITRz between nucleus and cytoplasm (Benedict et al 1998, Crone et al 1999). A further improvement was modification of the SNIP cassette, so that a short poly(A) track was present at the 3′-end of the liberated dITRz (SNIPAA); other alternative modifications included addition of 3′ histone mRNA binding region (SNIPHis) or a short hairpin loop (SNIPHP). All of these modifications resulted in stabilization of the liberated dITRz within cells. Using radiolabeled RT/PCR, the modifications to the dITRz resulted in relative increases of 2.6×, 2.5×, and 1.5×, comparing the SNIPAA, SNIPHIS, and SNIPHP dITRz with the concentration of the dITRz released from the original SNIP cassette (FIG. 9A). Identical figures were obtained with 2 different primer pairs. Use of SNIPAA had the additional benefit of increasing the catalytic activity of the dITRz in vitro by 30% (FIG. 9B). The Real Time RT/PCR results for the dITRz agreed reasonably well with the radiolabeled RT/PCR described above: Relative fluorescence intensity for the SNIPAA, SNIPHIS, and SNIPHP dITRz amplifications were 2.2×, 1.5×, and 1.9× compared with that from the SNIP cassette (respectively). The typical Ct (cycle threshold) values obtained for the liberated dITRz were approximately 26. Real-Time RT/PCR Ct values for the 18S rRNA control were 16.16+0.04. No amplification (at <40 cycles) of the various other regions was observed, suggesting that they were rapidly degraded (see FIG. 8). This supports previous data which suggested that all transcripts undergo autocatalytic processing within cells (Benedict et al 1998).

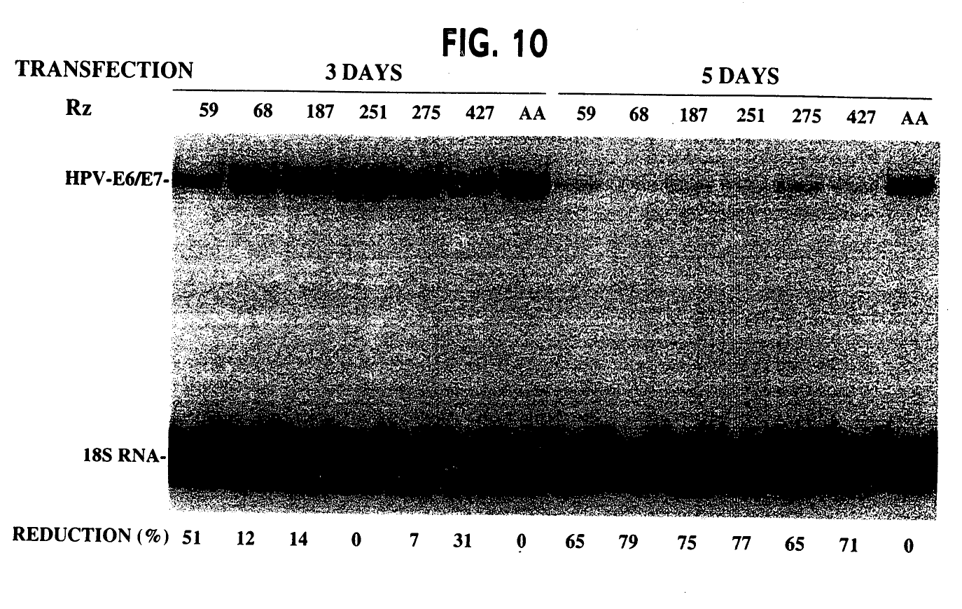

The HPV16-E6/E7 construct and the SNIPAARz construct were co-transfected into 293 cells, and cultures were analyzed for the effects of sRz on HPV16-E6/E7 RNA expression. At 3 and 5 days after transfection with the SNIPAA constructs containing sRzs (Rz59, Rz68, Rz187, Rz251, Rz275) or with SNIPAARz427 (the most active Rz previously identified using our modified SELEX procedure), a substantial reduction of HPV16-E6/E7 RNA was observed (FIG. 10). The largest reduction was produced by SNIPAAsRz59 at day 3, and by day 5, the E6/E7 transcript was barely detectable in all of the sRz transfections, compared with co-transfections with the empty SNIPAA cassette.

We also conducted preliminary growth experiments with SiHa cells. SiHa cells are a human cell line derived from a cervical squamous cell carcinoma. They contain an integrated copy of HPV16, and their growth is dependent upon continued expression of HPV16 E6/E7 transcripts (Madrigal et al 1997, Rorke 1997, Tan & Ting 1995). SiHa cells were transfected with the pCMV/BSD plasmids containing the various sRz in the SNIPAA cassette, and the transfected populations were selected for antibiotic resistance (with BSD at 10 μg/ml) on the plasmid. After 8 days, cells were counted (in triplicate samples). Compared with cells transfected with the empty SNIPAA cassette (or GFP), cells transfected with the previously identified SNIPAARz427 showed a modest 15% reduction in cell growth. Populations transfected with the 59, 68, or 251 SNIPAAsRz constructs showed 40-45% the largestreductions in cell growth (p<0.05), similar to reductions observed in other studies (Madrigal et al 1997, Tan & Ting 1995). Those transfected with the 187 or 275 SNIPAAsRz constructs showed growth rates only slightly reduced from control. Corresponding decreases were observed in endogenous E6/E7 transcript levels.

In summary, this library-screening procedure provides a rapid method for determining efficient cleavage sites in long, structured target RNAs. Re-amplification and transcription of selected Rz-library RNA pools has streamlined the procedure to 2 rounds of selection, and the entire procedure can be finished in a few days. Rz targeted to the identified regions have been shown to be very active in vitro, and the selected Rz targeted to HPV16 E6/E7 have also been shown to be efficacious in a cell culture models, demonstrating the utility of the Rz-library screening method in designing hammerhead Rz that are active in vivo.

Materials and Methods

Construction of DNA Library and Target RNA Templates

A single-stranded DNA library containing 6.87×10¹⁰ sequences (1.5 mg of DNA) was constructed by automated solid-state synthesis (Gibco BRL Custom Primers, Life Technologies). The sequence diversity was created by randomizing two domains totaling 18 nt (9 Ns and 9 Ns) flanking that Rz catalytic core (23 nt), and using fixed sequences for both 5′/3′-ends. The library sequence was 5′-CGCAGACCCTTGGAATTC-NNNNNNNNN-TTTCGTCCTCACGGACTCATCAG-NNNNNNNNN-GGATCCTGGAACCGACGAT-3′. The Sp6 primer (5′-GCCAAGCTATTTAGGTGACACTATAGATCGTCGGTTCCAGG-ATCC-3′, containing an Sp6 RNA polymerase promoter), 5′-end primer (5′-GCCAAGCTATTTAGGTGA-3′) and 3′-end primer (5′-CGCAGACCCTTGGAATTC-3′) were designed to utilize polymerase chain reaction (PCR) amplification of the randomized sequence in order to construct the double-stranded DNA library (FIG. 1B). The library was sequenced to confirm its composition (FIG. 1C). The library was then transcribed using Sp6 RNA polymerase to generate a random pool of multiple copies of approximately 70 billion different Rz sequences.

Target RNA pre-templates (no promoter and/or Rz tail) were produced by PCR for human papillomavirus type I (HPV11-E6/E7, 731 nt, accession # M14119), human immunodeficiency virus (HIV-TAT, 264 nt; accession # K03455), human malignant melanoma metastasis-suppressor (Kiss-1, 441 nt; accession # U43527), and mouse C9 subunit of the multicatalytic proteinase (MCP-C9, 1166 nt, accession # X53304; Ren et al., 1999).

Reverse transcription/PCR (RT/PCR) was used to generate the pre-template construct of Human papillomavirus type 16 E6/E7 (HPV16-E6/E7, 782 nt in length, accession # K02718), and human apoptosis inhibitor 4 (API4, 557 nt, accession # MN001168). This was performed using total RNA isolated from CaSki cells (a human cervical epidermoid carcinoma, ATCC CRL-1550). RNA was isolated with TRIzol Reagent (Gibco BRL), and the RT was performed using a modified method with the Super-ScriptTM II RNase H-Reverse Transcriptase (Gibco BRL). 1 μg of total RNA, in a volume of 12 μL 20 mM Tris-HCl (pH 7.4), was heated with 10 pmol of HPV16-E6/E7 RT-primer (5′-TTATGGTTTCTGAGAACAGAT-3′) or API4 RT-primer (5′-ACCCTGGAAGTGGTGCAGCCA-3′) at 85° C. for 3 min. The temperature was gradually decreased to 25° C. over 30 min, after which the other components were added according to manufacturer's instructions, and incubation was at 48° C. for 1 hr to generate cDNA of HPV16-E6/E7 or API4.

PCR-construction of pre-template DNA utilized Platinum Taq DNA Polymerase (Gibco BRL). The 5′-primers used were: 5′-ATGCACCAAAAGAGAACTGCA-3′ for HPV 16-E6/E7; 5′-ATGGAAAGTAAAGATGCCTCC-3′ for HPV11-E6/E7; 5′-ATGGAGCCAGTAGATCCTCGT-3′ for HIV-TAT; 5′-ATGAACTCACTGGTTTCTTGG-3′ for Kiss-1; 5′-CATGCCCCGCGGCGCGCCATT-3′ for API4; and 5′-CAGTTCTGCGCACGCGCGCGG-3′ for MCP-C9. The 3′-primer used for HPV16-E6/E7 and API4 were same as the RT-primers described above, the others were: 5′-CACTAGTAACGGCCGCCAGTG-3′ for HPV11-E6/E7; 5′-CCCTTCCTTCGGGCCTGTCGG-3′ for HIV-TAT; 5′-TCACTGCCCCGCACCTGCGCC-3′ for Kiss-1; and 5′-CAATCTTTCCAGGTTTTATTC-3′ for MCP-C9.

Double-stranded DNA templates for production of targeted RNA transcripts were constructed by adding the T7 RNA polymerase promoter (for all) and Rz tail (for transcripts about 700 nt), using PCR amplifications (FIG. 2). The T7-promoter primer was designed for adding the T7 RNA polymerase promoter to the 5′-end of the pre-template, and the Rz-primer added an additional tail at the 3′-end. The Hind III and Xba I restriction endonuclease sites were designed for advancing the PCR-construction and to allow facile cloning. The X-part of the T7 primer, 18 nt in length, was the sense sequence of the 5′-end of the pre-template (see FIG. 3). The Q-part of the Rz primer (18 nt) was the anti-sense sequence of 3′-end, and the P-part (about 8 nt) generated an 8 nt RNA that formed the 3′-end of Helix III. In a 50 μL of PCR reaction, 1 ng of the pre-template DNA was amplified with 10 pmol of T7/Rz primers and 100 pmol of Hind III/Xba I primers under standard PCR conditions.

To generate the selected Rz (sRz), sets of oligonucleotides were synthesized (Gibco BRL), which consisted of a central catalytic core domain of the hammerhead Rz (23 nt), flanked by two variable domains (9 Ns adjacent to the 5′-end of the core, and 6 Ns adjacent to the 3′-end, which would base pair in reverse complementary fashion with the target-RNA), and by fixed 5′/3′-end sequences. The overall design was therefore 5′-GACCCTTGGAATTC-9N-TTTCGTCCTCACGGACTCATCAG-6N-GGATCCTGGAACCCTATAG-3′ (FIG. 1A). The double stranded DNA templates for in vitro transcription were made by PCR with a 3′-end primer, 5′-GACCCTTGGAATTC-3′; and two 5′-end primers, 5′-GCCAAGCTATTTAGGTGACACTATAGGTTCCAGGATCC-3′ containing a T7 RNA polymerase promoter, and 5′-GCCAAGCTATTTAGG-3′ as an “accelerator” for PCR construction of the sRz-templates. All constructs were sequenced in their entirety prior to use.

Transcription of Library RNA and Target-RNA

Both the Rz-library RNA pool and target RNA were transcribed in vitro using the Riboprobe System (Promega) with ³²P-CTP; Sp6 (for Rz-library RNA) or T7 (for target-RNA) RNA polymerases were utilized and reactions were performed at 37° C. for 2 hr, followed by a digestion with RNase-Free DNase to destroy the template DNAs. The transcripts were extracted with phenol/chloroform, heated at 85° C. for 3 minutes in an equal volume of loading buffer (80% formamide, 100 mM EDTA, pH 8.0, 0.05% bromophenol blue, 0.05% xylene cyanol FF) and purified by PAGE (Benedict et al 1998). The corresponding bands were excised, homogenized in buffer (20 mM Tris-HCl, pH 7.4, 250 mM NaCl) and then incubated for 2 hr at 4° C. and then for 5 min at 85° C. Following centrifugation at 2000×g for 5 min the supernatant was recovered, the RNA was precipitated with ethanol and then resuspended in 20 mM Tris-HCl (pH 7.4).

In Vitro Rz-Library Screening

Two rounds of selection were performed for each target RNA (only 1 round was performed for HIV-TAT). Each round of selection was performed as follows: In a total volume of 100 μL reaction, 100 μM Rz-library RNA pool and 1 μM target-RNA were diluted with 20 mM Tris-HCl (pH 7.4), heated to 85° C. for 3 min and then cooled to 37° C. over a 30 minute period allowing RNA-RNA complexes to form. 1/5 volume of loading buffer (20% glycerol plus 0.05% bromophenol blue and 0.05% xylene cyanol FF) was added, and the bound complexes were separated from the unbound Rz-library RNA pool in a non-denaturing, 8% (urea-free) polyacrylamide-TBE gel. The RNA-RNA complexes (containing the bound species from the Rz library) were isolated and purified as described above, and resuspended in 20 mM Tris-HCl (pH 7.4). The selected Rz-library RNAs were reverse transcribed to produce their cDNAs using 3′-end primer (as described above for construction of Rz-library template) by employing Omniscript™ reverse transcriptase (Qiagen), subjected to PCR-amplification using Sp6 RNA polymerase promotor primer and 5′-/3′-end primers, and subsequently transcribed using Sp6 RNA polymerase to produce a new Rz library RNA pool which was enriched for better target-RNA-binding sequences for each specific target-RNA.

Each of these new Rz-library RNA pools was again selected using the corresponding target-RNA. For this second round selection stringency was increased by reducing (by half) the target-RNA concentration. After 2 rounds (1 round for HIV-TAT), the selected pools of Rz-library RNA were used to cleave the corresponding target-RNA.

To produce 5′-end ³²P-labeled target RNA, Alkaline Phosphatase (Calf Intestinal, 10 units/μL, New England Biolabs) was employed to remove the triphosphate groups from the 5′-end of unlabeled transcripts (³²P free); the dephosphorylated transcripts were then labeled using T4 polynucleotide kinase (10 units/μL, New England Biolabs) with γ-³²P-ATP. To produce 3′-end ³²P-labeled target RNA, which had a precise 3′-end produced by use of a 3′-cis-acting Rz, T4 polynucleotide kinase was employed to cleave the 2′/3′ cyclic phosphate bond and remove the phosphate group (Loria & Pan 2000), then the dephosphorylated transcripts were labeled by Poly (A) polymerase (500 units/μL, Amersham Life Science) with α-³²P-CoTP (BLU/NEG/026, DuPont).

In a volume of 5 μL reaction, a trace amount of end-labeled target RNA (about 50,000 cpm) was incubated with 10 μM of the selected Rz-library RNA pool in 20 mM Tris-HCl (pH 7.4) and 25 mM MgCl₂, at 37° C. for 2 hrs. The cleaved samples were analyzed by PAGE using a 6% urea gel, in comparison with A, G, and limited alkaline hydrolysis ladders (Donis-Keller 1980). The gel was dried, and then exposed to autoradiographic film.

In Vitro Cleavage Tests of Selected Rz

Rz targeted to the individual library-selected sites were transcribed from double-stranded DNA oligonucleotides, using Sp6 RNA polymerase as described for generation of the Rz-library RNAs. The size of the transcripts was exactly the same as the internal Rz liberated from the CHOP portion of the SNIPAA cassette (FIGS. 7 and 8A). For standard screening of Rz activity, incubations contained trace amounts of ³²P-labeled target RNA and 200 nM Rz RNA, and were for 1 hr at 37° C. in 20 mM Tris-HCl (pH 7.4), 25 mM MgCl₂. After incubations, samples were separated by PAGE in a 6% urea gel; the gels were then dried and radioactivity was analyzed using a PhosphorImager.

For kinetic analyses, a trace amount of ³²P-labeled target-RNA was mixed with unlabeled target-RNA (to yield final concentrations of 10, 100, 333 and 1000 nM target RNA) and Rz-RNA (100 nM final concentration), and incubations were performed using the same conditions as described previously (Pan et al 2001), except that incubation times were varied (for 40 sec, 1, 2, 5, 10, 30 min and 1 hr). The samples were then separated by PAGE in a 6% urea gel, and then dried and analyzed using a PhosphorImager.

Effects of sRz on HPV Expression in Cell Culture

To test the effectiveness of sRz in cell culture, a set of Rz cassettes named SNIP (FIG. 7) were constructed based on modification of the original CLIP cassette (Benedict et al 1998, Crone et al 1999). The double internal trans-acting ribozymes (dITRz) were effectively liberated in vitro (FIG. 8A) and in cell culture (FIG. 8B); Modifications to the dITRz included the addition of 3′-tails of poly A (AA), histone mRNA binding region (His), or a 10 bp hairpin-loop structure (HP). These modifications all significantly prevented RNA degradation within cells (FIG. 9A); and the modified dITRz still efficiently cleaved their long and structured target-RNA (FIG. 9B).

For RT/PCR analysis of co-transfection experiments, 293 cells were maintained in minimal essential medium supplemented with 10% heat-inactivated fetal bovine serum, in a humidified incubator at 37° C. with 5% CO₂. These cells were co-transfected with pVAx1 (InVitrogen) containing the HPV16-E6/E7 sequence, and either pCMV/BSD (also InVitrogen) containing SNIPAA, SNIPAARz59, SNIPAARz68, SNIPAARz187, SNIPAARz251, SNIPAARz275, or SNIPAARz427. The pCMV/BSD-SNIPAAsRz were constructed by annealing reverse complementary oligonucleotides, and then inserting them into the BglII/Mfe I sites of the CLIP portion of the cassette, and the BamH I/EcoR I sites of the CHOP portion of the cassette.

293T cells were transfected using LipofectAMINE transfection reagent (Life Technologies). A total of 2 μg of DNA (1 μg of pVAx1HPV16-E6/E7 and 1 μg of the pCMV/BSD-SNIPAAsRz constructs) in 12 μL of LipoFectAMINE and 8 μL of PLUS reagent was used to transfect a 60 mm dish of cells seeded 24 hours prior to transfection. The reagent/DNA mixture was incubated in Dulbecco's Modified Eagle Medium containing 5% bovine calf serum for 3 hours, and then adjusted to 10% serum for an additional 24 hours incubation.

For RT/PCR analyses, total RNA was isolated from transfected 293T cells three and five days post-transfection using RNAqueousTM-4PCR kits (AMBION), and followed by a DNase treatment. Reverse transcription was performed using 50 ng of total RNA with SensiscriptTM Reverse Transcriptase (QIAGEN). The ³²P-labeled PCR was performed using HotStarTaqTM DNA Polymerase (QIAGEN). One pair of primers for HPV16-E6/E7 (5′-GTCAAAAGCCACTGTGTCC-3′; 5′-ACAACCGAAGCGTAGGGTCA-3′) generated a 345 bp PCR product and another pair of primers for 18S rRNA (PE Applied Biosystems) generated a 186 bp PCR product. The products were separated by PAGE in urea-free gels, and then analyzed using a PhosphorImager (FIG. 10). Reactions were generally for 30 rounds, during which time amplification of HPV transcripts was in the linear range.

For real-time PCR studies, we used a Stratagene Mx4000 machine and TaqMan 5′-nuclease methodology, with 6-carboxy-fluorescein (FAM) and Black Hole Quencher 1 (BHQ). We chose a representative SNIPAARz construct for these analyses, SNIPAARz777/885, which contained dITRz targeted to Hepatitis B Virus. The TaqMan probe was (FAM)-ACGAAATTAGGCAGAAAACGACTGATGAGTC-(BHQ), which was specific for the liberated dITRz. 293T cells were transfected as described, and various primer pairs were used for real-time RT/PCR amplification of the various regions derived from the autocatalytic self-processing of the SNIPAA cassette (The locations of the primers within the SNIPAA cassette are shown schematically in FIG. 7). The primers used were as follows: RP3, GTTCCAAAGCTGGATATCCGCTGC; FP1, CGGTACCGTCAG CTCGACCTC; RP1, GCGGCCGCATAGGAACGCGT; FP3, CACGGTCAGCAGAATGTCATC; FP2, GATCCAGAGATCTGATGA; and RP2, AATTCTGGAGTTACTTTCGTCCTCACG, and the primer pairs used for amplification of the various regions are shown below.

TABLE 8

| | Region of SNIP | | | | | | | |
|---|---|---|---|---|---|---|---|---|
| | ITRz | S1 | S2 | S3 | S4 | CLIP | CHOP | SNIP |
| Primer | RT- | RT- | RT- | RT- | RT- | RT-RP3 | RT-RP1 | RT-RP1 |
| | RP2 | RP2 | RP3 | RP2 | RP1 | | | |
| Pairs | FP2 | FP1 | FP2 | FP3 | FP2 | FP1 | FP1 | FP1 |
| | RP2 | RP2 | RP3 | RP2 | RP1 | RP3 | RP1 | FP1 |

As internal controls, RT/PCR amplification of 18S rRNA was performed in the same samples, using VIC-labeled primer (Applied Biosystems). The Ct value for the 18S rRNA was 16.16+0.04. Finally, ROX (carboxy-X-rhodamine, succinimidyl ester) was used as dye for a volume control.

In other experiments SiHa cells were used in growth studies. Rz427 has previously been shown to significantly inhibit growth of CaSki (a human cervical epidermoid carcinoma, ATCC CRL-1550) and SiHa (a human cervical squamous cell carcinoma, ATCC HTB-35) cell lines. Both of these cell lines contain integrated HPV16, and their growth is at least partially dependent upon continued production of the E6/E7 transcript.

SiHa cells were transfected with 3 μg of pCMV/BSD plasmid (InVitrogen) containing the various SNIPAAsRz constructs, using the Effectine transfection reagent (Qiagen). The cells were maintained in Minimum Essential Medium Alpha (GibcoBRL) with 10% bovine calf serum, also containing 10 μg/ml Blasticidin S (BSD) antibiotic, to select for successfully transfected cells using BSD deaminase activity. This selection was complete after 6 days. After 8 days, cells were counted in triplicate samples. In various experiments, transfections were also scaled up to allow for RNA isolation, as described above, and Northern blot analysis.

TABLE 9
Summary of Identified Cleavage Sites.

| | | | | |
|---|---|---|---|---|
| | | | Cleavage | |
| Target | Accession # | sRz | Site | Target Sequence |

| | | | | |
|---|---|---|---|---|
| HPV16 | K02718 | 59 | 138 | CCCAGAAAGUUACCACA |
| | | 68 | 147 | UUACCACAGUUAUGCAC |
| | | 150 | 229 | GACGUGAGGUAUAUGAC |
| | | 187 | 266 | CAUAGUAUAUAGAGAUG |
| | | 251 | 330 | AUUAGUGAGUAUAGACA |
| | | 275 | 354 | UAUAGUUUGUAUGGAAC |
| | | 415 | 494 | UAUAAGGGGUCGGUGGA |
| | | 427 | 506 | GUGGACCGGUCGAUGUA |
| HPV11 | M14119 | 162 | 260 | ACCUAAAGGUUGUGUGG |
| | | 244 | 342 | UAGACACUUUAAUUAUG |
| | | 409 | 507 | GUGGAAGGGUCGUUGCU |
| | | 449 | 547 | GAAGACUUGUUACCCUA |
| | | 499 | 597 | CCUGUAGGGUUACAUUG |
| | | 529 | 627 | GAAGACAGCUCAGAAGA |
| | | 593 | 691 | AUUACCAAAUACUGACC |
| HIV-TAT | K03455 | 82 | 5909 | CAAUUGCUAUUGUAAAA |
| | | 190 | 6017 | UCAGAACAGUCAGACUC |
| | | 208 | 6035 | UCAAGCUUCUCUAUCAA |
| Kiss-1 | U43257 | 202 | 410 | GCUGAGCCGUCGGGGGA |
| API4 | MN001168 | 252 | 226 | AGUGUUUCUUCUGCUUC |
| | | 353 | 327 | AAGAAGCAGUUUGAAGA |
| MCP-C9 | X53304 | 106 | 103 | AGCCAUGUCUCGAAGAU |
| | | 148 | 145 | UCCAGAAGGUCGCUUAU |

TABLE 10
Analysis of Nucleotide Positions Surrounding
Identified Cleavage Sites.

| | Position | | | | | | | | | | | | | | | | |
|---|---|---|---|---|---|---|---|---|---|---|---|---|---|---|---|---|---|
| | III.10 | III.9 | III.8 | III.7 | III.6 | III.5 | III.4 | III.3 | N | U | H | I.1 | I.2 | I.3 | I.4 | I.5 | I.6 |
| Adenosine | 6 | 9 | 6 | 10 | 10 | 8 | 7 | 6 | 3 | | 6 | 7 | 6 | 6 | 9 | 7 | 10 |
| Cytidine | 4 | 7 | 5 | 2 | 3 | 5 | 7 | 2 | 3 | | 10 | 1 | 4 | 3 | 4 | 4 | 6 |
| Guanosine | 6 | 2 | 4 | 9 | 6 | 5 | 5 | 8 | 15 | | | 9 | 7 | 7 | 6 | 5 | 4 |
| Uridine | 7 | 5 | 8 | 2 | 4 | 5 | 4 | 7 | 2 | 23 | 7 | 6 | 6 | 7 | 4 | 7 | 3 |

### Example 6

### Application of Library Selection Technology to “Real-time” or Quantitative PCR

Real-time PCR, or quantitative PCR (qPCR), is a relatively new technology for quantitatively assessing nucleic acid levels in samples. It represents a reverse transcription/PCR amplification from starting RNA samples. The initial 3′ primer is used in the reverse transcription reaction. The 5′ primer is then used in conjunction with the 3′ primer for PCR amplification cycles. The middle primer is labeled with a fluorescent dye. A 5′ nuclease activity of the TaqMan polymerase is used in the PCR step, which degrades the middle primer, and the fluorescent probe is released and produces fluorescence which is measured each cycle (i.e., real-time). This 3-primer arrangement also provides much better specificity, since only products encompassing all 3 primers will ultimately produce fluorescence.

Primers for qPCR are chosen using a software program called “Primer Express” (from PE Applied Biosystems). It is based on linear sequence comparisons and properties. However, the RT step is run at 37° C. or 42° C., and the accessibility of the chosen primer site is important. Furthermore, not all 5′/3′ primer pairs chosen work well in the PCR amplification steps.

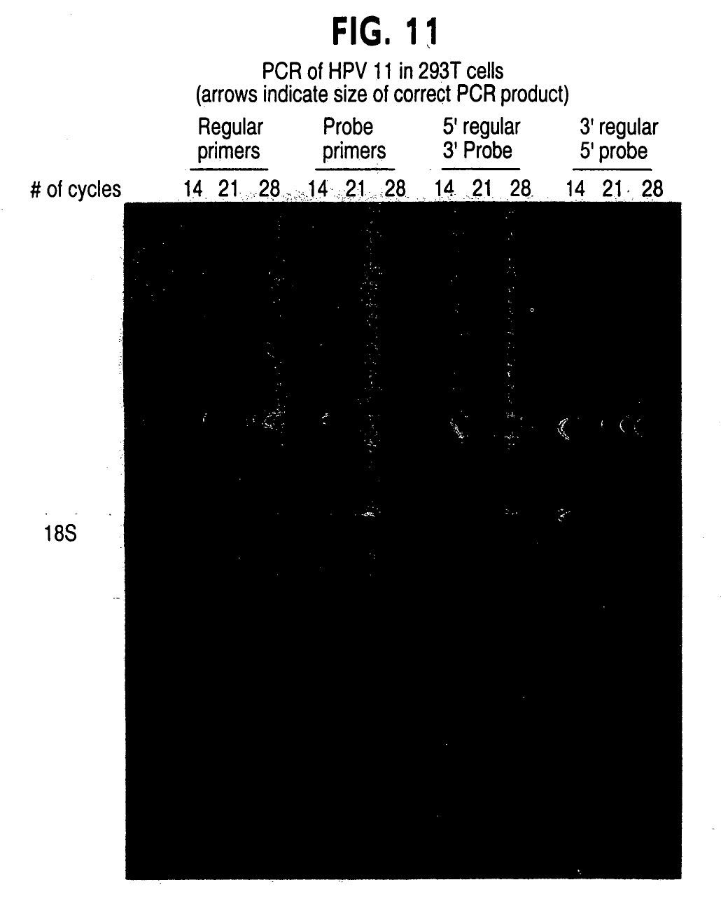

In studies for qPCR analyses of HPV11 E6/E7 mRNA transcripts, we have compared amplifications using a primer chosen using Primer Express, to amplifications using a primer located at a library-selected accessible site. 5′ regular (C634) and 3′ regular (C1124) primers were located near the ends of the in vitro transcribed HPV11 E6/E7 transcripts. A 5′ probe primer (nt 154-175) was placed in an accessible region identified by library selection. A 3′ probe primer (the reverse complement of nt 226-203) was chosen using Primer Express. Standard radiolabeled RT/PCR amplifications showed that generation of PCR products was approximately 20 times greater with the library-selected primer location compared with the Primer Express-selected location (see FIG. 11). Real-time PCR was also performed with the specified primers (see Table 11). The results showed that when a library-selected primer was utilized with the 3′-probe primer, the Ct value was reduced by 5.7, compared with that obtained using the 5′-regular primer. This yields an increase in amplification (i.e. detection) of 50 times using a library-selected primer region versus a non-selected primer region (see Table 12).

By library-selecting accessible sites, one can clearly increase the sensitivity of the detection of targeted mRNA transcripts.

TABLE 11

HPV11-E6/E7 Region; Sequence and primer locations for
Real-time PCR (Complementary Strand not shown)

gggatggaaa gtaaagatac ctccacgtct gcaacatcca tagaccagtt gtgcaagacg
tttaatcttt ctttgcacac tctgcaaatt cagtgcgtgt tttgcaggaa tgcactgacc
accgcagaga tatatgcata tgcctataag aacctaaagg ttgtgtggcg agacaacttt
ccctttgcag cgtgtgcctg ttgcttagaa ctgcaaggga aaattaacca atatagacac
tttaattatg ctgcatatgc acctacagta gaagaagaaa ctaatgaaga tattttaaaa
gtgttaattc gttgttacct gtgtcacaag ccgttgtgtg aaatagaaaa actaaagcac
atattggaaa aggcacgctt cataaaacta aataaccagt ggaagggtcg ttgcttacac
tgctggacaa catgcatgga agacttgtta ccctaaagga tatagtacta gacctgcagc
ctcctgaccc tgtagggtta cattgctatg agcaattaga agacagctca gaagatgagg
tggacaaggt ggacaaacaa gactcacagc ctttaacaca acattaccaa atactgacct
gttgctgtgg atgtgacagc aacgtccgac tggttgtgga gtgcacagac ggagacatna
gacaactaca agaccttttg ctgggcacac taaatattgt gtgtcccatc tgcgcaccaa
aaccataaca agggcgaatt ccagcacact ggcggccgtt actagtg

Primer locations and complementary sequences are underlined

Regular Primers

| | |
|---|---|
| 5′ primer (C634; | 5′-ATGGAAAGTAAAGATGCCTCCAC-3′ |
| nt 102-124): | |
| 3′ primer (C1124; | 5′-CCCATCTGCGCACCAAAACCATAAC-3′ |
| nt 827-803): | |

Probe Primers

| | |
|---|---|
| 5′ primer | 5′-CTAAAGGTTGTGTGGCGAGACA-3′ |
| (nt 154-175): | |
| 3′ primer | 5′-GCTTAGAACTGCAAGGGAAAATTAA-3′ |
| (nt 226-203): | |

| | |
|---|---|
| Probe (nt 177-201): | 5′-CTTTCCCTTTGCAGCGTGTGCCTGT-3′ |

Radiolabeled PCR: test with different pairs of primers (or their complements, between regular and probe primers).

a) Followed standard procedure PCR for HotStar Taq Poly.

b) Cycles run: 14, 21, 28.

c)

| | | | |
|---|---|---|---|
| Temperature: | 95° C. | 15 | min |
| | 95° C. | 1 | min |
| | 52° C. | 1 | min |
| | | 30 | s |
| | 72° C. | 3 | min |
| | 72° C. | 10 | min |

Then cast PCR products on 6% native PAGE until the first dye reached 80% gel.

Dried gel for 40 min and exposed to the X-ray film overnight.

Results:

The combination of 5′ probe primer and 3′ regular primer showed the sharpest and strongest bands of PCR products of HPV11 and control 18S without nonspecific bands as others at 21 cycles (see FIG. 11).

TABLE 12
HPV 11 IN 293T CELLS: PRIMER TEST USING QPCR

| Treatments | Ct ± SEM (3 Exp) |
|---|---|
| No Template Control | 42.6 ± 2.44 |
| No HPV11 primers | No Ct |
| 293T cell line | No Ct |
| Plasmid HPV11 in pVAX1 | 25.9 ± 0.4 |
| HPV11 in 293T-day 1 after | |
| transfection | |
| 5′ probe + 3′ probe (˜87 bp) | 20.1 ± 0.15 |
| 5′ regular + 3′ probe (˜224 bp) | 25.8 ± 0.1 |

### Example 7

### Removal of Extraneous Flanking Sequences from Library Selected Ribozymes

A number of ribozymes targeted to library selected sites have been tested, either without extraneous flanking sequences, or with the extraneous flanking sequences which are present in the library Rz. The result is that those without flanking sequences are about 24 times more active than those contained within the library pool. Removing extraneous flanking sequences increases activity considerably because when cut products are detected after incubation with the Rz library pool, the actual Rz subsequently designed are more active against the sites.

When the Rz library is transcribed in vitro, the transcript includes the fixed 5′ and 3′ sequences described previously herein under Library Construction. When the cutting sites are identified, the corresponding random sequences in the ribozymes are also defined, this is also described herein under Library Construction. When the sRz is subsequently constructed, the sRz is made containing the newly identified (previously random) flanking sequences surrounding the catalytic core. However, for the Rz which actually did the cutting in the library pool, the extraneous fixed sequences were present. sRz without the fixed sequences are about 24 times more active (on average) than the same sRz containing the fixed sequences. Therefore, when active Rz are identified in the pool, the sRz that are subsequently constructed are much more active.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

### References

Amarzguioui M, Prydz H. 1998. Hammerhead ribozyme design and application. Cell Mol Life Sci 54: 1175-202.

Benedict C M, Pan W, Loy S E, Clawson G A. 1998. Triple ribozyme-mediated down-regulation of the retinoblastoma gene. Carcinogenesis 19: 1223-30.

Birikh K, Heaton P, Eckstein F. 1997a. The structure, function and application of the hammerhead ribozyme. Eur.J.Biochem. 245: 1-16

Birikh K R, Berlin Y A, Soreq H, Eckstein F. 1997b. Probing accessible sites for ribozymes on human acetylcholinesterase RNA. RNA 3: 429-37.

Campbell T B, McDonald C K, Hagen M. 1997. The effect of structure in a long target RNA on ribozyme cleavage efficiency. Nucleic Acids Res 25: 4985-93.

Clouet-d'Orval B, Uhlenbeck O. 1997. Hammerhead ribozymes with a faster cleavage rate. Biochemistry 36: 9087-92.

Crone T M, Schalles S L, Benedict C M, Pan W, Ren L, et al. 1999. Growth inhibition by a triple ribozyme targeted to repetitive B2 transcripts. Hepatology 29: 1114-23.

Donis-Keller J. 1980. Phy M: An RNase activity specific for U and A residues useful in RNA sequence analysis. Nucl. Acids Res. 8: 3133-42

Eldadah B A, Ren R F, Faden A I. 2000. Ribozyme-mediated inhibition of caspase-3 protects cerebellar granule cells from apoptosis induced by serum-potassium deprivation. J Neurosci 20: 179-86.

Folini M, Colella G, Villa R, Lualdi S, Daidone M G, Zaffaroni N. 2000. Inhibition of telomerase activity by a hammerhead ribozyme targeting the RNA component of telomerase in human melanoma cells. J Invest Dermatol 114: 259-67.

Gaughan D J, Whitehead A S. 1999. Function and biological applications of catalytic nucleic acids. Biochim Biophys Acta 1445: 1-20.

Goodchild J, Kohli V. 1991. Ribozymes that cleave an RNA sequence from human immunodeficiency virus: the effect of flanking sequence on rate. Arch Biochem Biophys 284: 386-91.

Hendry P, McCall M. 1996. Unexpected anisotropy in substrate cleavage rates by asymmetric hammerhead ribozymes. Nucl. Acids Res. 24: 2679-84.

James H, Gibson 1. 1998. The therapeutic potential of ribozymes. Blood 91: 371-82.

Jen K Y, Gewirtz A M. 2000. Suppression of gene expression by targeted disruption of messenger RNA: available options and current strategies. Stem Cells 18: 307-19.

Lieber A, Kay M A. 1996. Adenovirus-mediated expression of ribozymes in mice. J Virol 70: 3153-8.

Lieber A, Strauss M. 1995. Selection of efficient cleavage sites in target RNAs by using a ribozyme expression library. Mol.Cell.Biol. 15: 540-51.

Lilley D M. 1999. Structure, folding and catalysis of the small nucleolytic ribozymes. Curr Opin Struct Biol 9: 330-8.

Loria A, Pan T. 2000. The 3′ substrate determinants for the catalytic efficiency of the Bacillus subtilis RNase P holoenzyme suggest autolytic processing of the RNase P RNA in vivo. Rna 6: 1413-22.

Macejak D G, Jensen K L, Jamison S F, Domenico K, Roberts E C, et al. 2000. Inhibition of hepatitis C virus (HCV)-RNA-dependent translation and replication of a chimeric HCV poliovirus using synthetic stabilized ribozymes. Hepatology 31: 769-76.

Madrigal M, Janicek M, Sevin B, Perras J, Estape R, et al. 1997. In vitro antigene therapy targeting HPV-16 E6 and E7 in cervical carcinoma. Gyn.Onc. 64: 18-25.

Nedbal W, Sczakiel G. 1997. Hammerhead ribozyme activity in the presence of low molecular weight cellular extract. Antisense Nucleic Acid Drug Dev 7: 585-9.

Pan W-H, Devlin H, Kelley C, Isom H, Clawson G. 2001. A selection system for identifying accessible sites in target RNAs. RNA 7: 610-21.

Parker R, Muhlrad D, Deshler J, Taylor N, Rossi J. 1992. Ribozymes: Principles and designs for their use as antisense and therapeutic agents. In Gene Regulation: Biology of Antisense RNA and DNA, ed. R Erickson, J Izant, pp. 55-70. New York: Raven Press.

Passman M, Weinberg M, Kew M, Arbuthnot P. 2000. In situ demonstration of inhibitory effects of hammerhead ribozymes that are targeted to the hepatitis Bx sequence in cultured cells. Biochem Biophys Res Commun 268: 728-33.

Perlman H, Sata M, Krasinski K, Dorai T, Buttyan R, Walsh K. 2000. Adenovirus-encoded hammerhead ribozyme to Bcl-2 inhibits neointimal hyperplasia and induces vascular smooth muscle cell apoptosis. Cardiovasc Res 45: 570-8.

Ren L, Schalles j, Isom C, Loy S, Benedict C, Clawson G. 1998. Construction and deployment of triple ribozymes targeted to multicatalytic proteinase subunits C3 and C9. Gene Therapy and Mol.Biol. 3: 1-13.

Rorke E. 1997. Antisense human papillomavirus (HPV) E6/E7 expression, reduced stability of epidermal growth factor, and diminished growth of HPV-positive tumor cells. J.Natl.Can.Inst. 89: 1243-6.

Salmi P, Sproat B S, Ludwig J, Hale R, Avery N, et al. 2000. Dopamine D(2) receptor ribozyme inhibits quinpirole-induced stereotypy in rats. Eur J Pharmacol 388: R1-2.

Sun L Q, Cairns M J, Saravolac E G, Baker A, Gerlach W L. 2000. Catalytic nucleic acids: from lab to applications. Pharmacol Rev 52: 325-47.

Suzuki T, Anderegg B, Ohkawa T, Irie A, Engebraaten O, et al. 2000. Adenovirus-mediated ribozyme targeting of HER-2/neu inhibits in vivo growth of breast cancer cells. Gene Ther 7: 241-8.

Symons R H. 1992. Small catalytic RNAs. Annu Rev Biochem 61: 641-71.

Tan T, Ting R. 1995. In vitro and in vivo inhibition of human papillomavirus type 16 E6 and E7 genes. Cancer Res. 55: 4599-605.

Zuker M, Stiegler P. 1981. Optimal computer folding of large RNA sequences using thermodynamics and auxiliary information. Nucleic Acids Res 9: 133-48.

What is claimed is:

1. A method of identifying one or more cleavage sites in a target RNA which are accessible to a ribozyme, said method comprising:
(a) generating a library of RNAs, wherein each RNA in said library comprises a catalytically active hammerhead ribozyme core, wherein said ribozyme core is flanked on each side by random nucleotide regions, wherein said random nucleotide regions are flanked on each side by fixed sequences which allow amplification and a sequence which allows transcription of said RNA;
(b) contacting said target RNA with said library of RNAs under conditions in which said ribozyme core is not catalytically active;
(c) separating RNAs that bind to said target RNA from RNAs that do not bind;
(d) generating an enriched library of RNAs comprising RNAs bound in step (c);
(e) repeating steps (a) through (d) at least one additional time with a reduced ratio of said target RNA to said library of RNAs;
(f) generating 5′ or 3′ end-labeled target RNA;
(g) contacting said 5′ or 3′ end-labeled target RNA of step (f) with an enriched library of RNAs of step (e) under conditions in which said ribozyme core of said library of RNAs is catalytically active such that said target RNA is cleaved to produce cleavage products;
(h) separating said cleavage products from step (g) and determining the sequence or sequences at which cleavage of said end-labeled target RNA occurred as a result of incubation of said end-labeled target RNA with said library of RNAs.

2. A method of claim 1, wherein said target RNA of step (f) is 3′ end-labeled target RNA.

3. A method of claim 2, wherein said 3′ end-labeled target RNA is of uniform length and is produced by a method comprising:
(a) constructing a target RNA containing a 3′ cis-acting catalytic ribozyme having a 3′ flanking sequence that is reverse complementary to the 3′ end of said target RNA;
(b) cleaving said target RNA at the 3′ end with said 3′ cis-acting catalytic ribozyme; and
(c) labeling said target RNA at the 3′ end produced in step (b);
wherein said target RNA labeled in step (c) is 3′ end-labeled target RNA of uniform length.

4. A method of claim 1, wherein said random nucleotide regions in step (a) are about six to about twelve nucleotides in length.

5. A method of claim 1, wherein said random nucleotide regions in step (a) are about seven to about eleven nucleotides in length.

6. A method of claim 1, wherein said random nucleotide regions in step (a) are about eight to about ten nucleotides in length.

7. A method of claim 1, wherein said random nucleotide regions in step (a) are about nine nucleotides in length.

8. A method of claim 1, wherein said sequence which allows transcription of said RNA is an Sp6 RNA promoter.

9. A method of claim 1, wherein said condition in which said ribozyme core is not catalytically active of step (b) is in the absence of Mg²⁺.

10. A method of claim 1, wherein said separating of step (c) is performed using electrophoretic chromatography or column chromatography.

11. A method of claim 1, wherein said enriched library of RNAs of step (d) is generated by PCR amplification of said RNA that binds in step (c).

12. A method of claim 1, step (e), wherein said repeating steps (a) through (d) is done at least two additional times.

13. A method of claim 1, step (e), wherein said repeating steps (a) through (d) is done at least three additional times.

14. A method of claim 1, step (e), wherein said repeating steps (a) through (d) is done at least four additional times.

15. A method of claim 1, wherein said conditions in which said ribozyme core of said library of RNAs is catalytically active of step (g) is in the presence of Mg²⁺.

16. A method of claim 1, step (h), wherein said separating of said cleavage products and determining the sequence or sequences at which cleavage of said target RNA occurred is done on a single polyacrylamide gel.

17. A method of claim 1, wherein said target RNA is modified to comprise a promoter.

18. A method of claim 17, wherein said promoter is a T7 RNA polymerase promoter.

19. A method of making a catalytically active ribozyme that is specific for a target RNA and accessible to a cleavage site on said target RNA comprising:
(a) identifying a cleavage site on a target RNA using a method of claim 1;
(b) constructing a ribozyme comprising a sequence that is complementary to a cleavage site of step (a).

20. A catalytically active ribozyme produced by a method of claim 19.

21. A method of identifying one or more potential sites in a target RNA which are accessible to an antisense oligonucleotide, wherein said method comprises:
(a) generating a library of antisense oligonucleotides, wherein each antisense oligonucleotide of the library comprises regions of random nucleotides flanked by fixed sequences which allow reamplification and transcription;
(b) contacting said target RNA with the library of antisense oligonucleotides;
(c) separating antisense oligonucleotides that bind to said target RNA from antisense oligonucleotides that do not bind;
(d) generating an enriched library of antisense oligonucleotides comprising antisense oligonucleotides bound in step (c);
(e) repeating steps (a) through (d) at least four times to obtain selected antisense oligonucleotides;
(f) sequencing the selected antisense oligonucleotides of step (e); and
(g) comparing the sequences determined in step (f) with the sequence of said target RNA to identify one or more potential sites in said target RNA which are accessible to an antisense oligonucleotide.

22. A method of making an antisense oligonucleotide that is accessible to a site in a target RNA comprising:
(a) identifying a site in a target RNA using a method of claim 21;
(b) constructing an antisense oligonucleotide comprising a sequences that is complementary to a site identified in step (a); wherein said antisense oligonucleotide of step (b) binds to and is accessible to a target RNA.

23. An antisense oligonucleotide made by a process of claim 22.

24. A method of conducting real-time PCR comprising labeling an antisense oligonucleotide of claim 23 with a detectable label to generate a labeled probe and using said labeled probe in a real-time PCR amplification.

25. A method of conducting an assay with a fixed polynucleotide array comprising labeling an antisense oligonucleotide of claim 23 with a detectable label to generate a labeled probe and using said labeled probe in an assay with a fixed polynucleotide array.

26. A method of identifying one or more cleavage sites in a target RNA which are accessible to a DNAzyme, said method comprising:
(a) generating a library of DNAzymes, wherein each DNAzyme in said library comprises a catalytically active DNAzyme core, wherein said DNAzyme core is flanked on each side by random nucleotide regions, wherein said random nucleotide regions are limited to no more than seven random nucleotides upstream of said DNAzyme core and no more than eight random nucleotides downstream of said DNAzyme core, wherein said random nucleotide regions are flanked on each side by fixed sequences which allow amplification;
(b) contacting said target RNA with said library of DNAzymes in the absence of Mg²⁺ such that said DNAzyme core is not catalytically active;
(c) separating DNAzymes that bind to said target RNA from DNAzymes that do not bind to said target RNA using a non-denaturing polyacrylamide gel;
(d) generating an enriched library of DNAzymes comprising amplifying by PCR DNAzymes bound in step (c) using two amplification primers, followed by unidirectional PCR amplification using a single primer to generate single stranded DNAzymes;
(e) generating 5′ or 3′ end-labeled target RNA;
(f) contacting said 5′ or 3′ end-labeled target RNA of step (e) with an enriched library of DNAzymes of step (d) under conditions in which said DNAzyme core of said library of DNAzymes is catalytically active such that said target RNA is cleaved to produce cleavage products;
(g) separating said cleavage products from step (f) and determining the sequence or sequences at which cleavage of said end-labeled target RNA occurred as a result of incubation of said end-labeled target RNA with said library of DNAzymes.

27. A method of making a catalytically active DNAzyme that is specific for a target RNA and accessible to a cleavage site on said target RNA comprising:
(a) identifying a cleavage site on a target RNA using a method of claim 26;
(b) constructing a DNAzyme comprising a sequence that is complementary to a cleavage site of step (a).

28. A DNAzyme produced by a method of claim 27.


SEQUENCE LISTING (104 sequences)

SEQ ID NO: 1 - RNA, 41 nt, Artificial Sequence
  exemplary sequence
nnnnnnnnncugaugaguccgugaggacgaaannnnnnnnn

SEQ ID NO: 2 - DNA, 18 bp, Artificial Sequence
  primer
gccaagctatttaggtga

SEQ ID NO: 3 - DNA, 45 bp, Artificial Sequence
  primer
gccaagctatttaggtgacactatagatcgtcggttccaggatcc

SEQ ID NO: 4 - DNA, 78 bp, Artificial Sequence
  library sequence
cgcagacccttggaattcnnnnnnnnntttcgtcctcacggactcatcagnnnnnnnnnggatcctggaaccgacgat

SEQ ID NO: 5 - DNA, 18 bp, Artificial Sequence
  primer
cgcagacccttggaattc

SEQ ID NO: 6 - DNA, 104 bp, Artificial Sequence
  library sequence
gccaagctatttaggtgacactatagatcgtcggttccaggatccnnnnnnnnnctgatgagtccgtgaggacgaaannnnnnnnngaattccaagggtctgcg

SEQ ID NO: 7 - DNA, 104 bp, Artificial Sequence
  library sequence
cgcagacccttggaattcnnnnnnnnntttcgtcctcacggactcatcagnnnnnnnnnggatcctggaaccgacgatctatagtgtcacctaaatagcttggc

SEQ ID NO: 8 - RNA, 79 nt, Artificial Sequence
  synthetically generated sequence
gaucgucgguuccaggauccnnnnnnnnncugaugaguccgugaggacgaaannnnnnnnngaauuccaagggucugcg

SEQ ID NO: 9 - RNA, 76 nt, Artificial Sequence
  exemplary sequence
nnnnnnnnnnnnnnnnnngucuacuagaaaguaguacugaugaggccgaaaggccgaacnnnnnnnnucuagagca

SEQ ID NO: 10 - DNA, 18 bp, Artificial Sequence
  primer
ccgaagcttaatacgact

SEQ ID NO: 11 - DNA, 46 bp, Artificial Sequence
  primer
ccgaagcttaatacgactcactatagggnnnnnnnnnnnnnnnnnn

SEQ ID NO: 12 - DNA, 77 bp, Artificial Sequence
  primer
gctctagannnnnnnngtttcggcctttcggcctcatcagtactactttcgtagtagacnnnnnnnnnnnnnnnnnn

SEQ ID NO: 13 - DNA, 18 bp, Artificial Sequence
  primer
tgctctagannnnnnnng

SEQ ID NO: 14 - RNA, 70 nt, Artificial Sequence
  exemplary sequence
nnnnnnnnnngucuacuacaacguaguacugaugaggccgaaaggccgaaannnnnnnnnnucuagagca

SEQ ID NO: 15 - DNA, 48 bp, Artificial Sequence
  library sequence
gcctctagagtcgaannnnnnnnntcnnnnnnagtgttcttcagtccc

SEQ ID NO: 16 - DNA, 16 bp, Artificial Sequence
  primer
gcctctragagtcgaa

SEQ ID NO: 17 - DNA, 38 bp, Artificial Sequence
  primer
ccgaagcttaatacgactcactatagggactgaacact

SEQ ID NO: 18 - DNA, 36 bp, Artificial Sequence
  primer
gacgagaagctttcagacttttgtaatttgtgtagt

SEQ ID NO: 19 - DNA, 51 bp, Artificial Sequence
  primer
ccgaagcttaatacgactcactatagggcatgtattcaatctaagcaggct

SEQ ID NO: 20 - DNA, 48 bp, Artificial Sequence
  primer
ccgaagcttaatacgactcactatagggggcttctcaggcggtggagg

SEQ ID NO: 21 - DNA, 54 bp, Artificial Sequence
  primer
ccgcactatttaggtgacagtatagaagcttatgacagccatcatcaaagagat

SEQ ID NO: 22 - DNA, 24 bp, Artificial Sequence
  primer
actgaaggaaagaagtcagaaggc

SEQ ID NO: 23 - DNA, 20 bp, Artificial Sequence
  primer
tcagtccatctcaactgcat

SEQ ID NO: 24 - DNA, 14 bp, Artificial Sequence
  synthetically generated oligonucleotide
gacccttggaattc

SEQ ID NO: 25 - DNA, 23 bp, Artificial Sequence
  synthetically generated oligonucleotide
tttcgtcctcacggactcatcag

SEQ ID NO: 26 - DNA, 19 bp, Artificial Sequence
  synthetically generated oligonucleotide
ggatcctggaaccctatag

SEQ ID NO: 27 - DNA, 15 bp, Artificial Sequence
  synthetically generated oligonucleotide
ttctcgggggcttgg

SEQ ID NO: 28 - DNA, 15 bp, Artificial Sequence
  synthetically generated oligonucleotide
gggcgcacctcttta

SEQ ID NO: 29 - DNA, 15 bp, Artificial Sequence
  synthetically generated oligonucleotide
tctgcctaaatctct

SEQ ID NO: 30 - DNA, 15 bp, Artificial Sequence
  synthetically generated oligonucleotide
tggagttactcgttt

SEQ ID NO: 31 - DNA, 15 bp, Artificial Sequence
  synthetically generated oligonucleotide
cgcagcaggtggagc

SEQ ID NO: 32 - DNA, 15 bp, Artificial Sequence
  synthetically generated oligonucleotide
cgcgggacgctttgt

SEQ ID NO: 33 - DNA, 15 bp, Artificial Sequence
  synthetically generated oligonucleotide
tcgcaatgtcatact

SEQ ID NO: 34 - DNA, 15 bp, Artificial Sequence
  synthetically generated oligonucleotide
tcatgctgacccgtc

SEQ ID NO: 35 - DNA, 15 bp, Artificial Sequence
  synthetically generated oligonucleotide
ccatgctgcaaagat

SEQ ID NO: 36 - DNA, 15 bp, Artificial Sequence
  synthetically generated oligonucleotide
atatcctcagctcag

SEQ ID NO: 37 - DNA, 15 bp, Artificial Sequence
  synthetically generated oligonucleotide
tgaagaccaacccac

SEQ ID NO: 38 - DNA, 15 bp, Artificial Sequence
  synthetically generated oligonucleotide
tttattgcaggggca

SEQ ID NO: 39 - DNA, 16 bp, Artificial Sequence
  synthetically generated oligonucleotide
aaaagggagacaattt

SEQ ID NO: 40 - DNA, 15 bp, Artificial Sequence
  synthetically generated oligonucleotide
atatattcccaattc

SEQ ID NO: 41 - DNA, 14 bp, Artificial Sequence
  synthetically generated oligonucleotide
gtagagttcccaca

SEQ ID NO: 42 - DNA, 15 bp, Artificial Sequence
  synthetically generated oligonucleotide
cagaaagacgaaggt

SEQ ID NO: 43 - DNA, 15 bp, Artificial Sequence
  synthetically generated oligonucleotide
ggaacaatagatgat

SEQ ID NO: 44 - DNA, 15 bp, Artificial Sequence
  synthetically generated oligonucleotide
gcaaattttaaggca

SEQ ID NO: 45 - DNA, 15 bp, Artificial Sequence
  synthetically generated oligonucleotide
gtggtgataaaagta

SEQ ID NO: 46 - DNA, 15 bp, Artificial Sequence
  synthetically generated oligonucleotide
tgagagacaataaca

SEQ ID NO: 47 - DNA, 15 bp, Artificial Sequence
  synthetically generated oligonucleotide
tagaacttaaaaccc

SEQ ID NO: 48 - DNA, 15 bp, Artificial Sequence
  synthetically generated oligonucleotide
atttgtgcatttatt

SEQ ID NO: 49 - DNA, 15 bp, Artificial Sequence
  synthetically generated oligonucleotide
tactcacccacaaaa

SEQ ID NO: 50 - DNA, 71 bp, Artificial Sequence
  synthetically generated oligonucleotide
gacccttggaattcnnnnnnnnntttcgtcctcacggactcatcagnnnnnnggatcctggaaccctatag

SEQ ID NO: 51 - DNA, 39 bp, Artificial Sequence
  primer
ccgaagcttaatacgactcactatagggttccaggatcc

SEQ ID NO: 52 - DNA, 61 bp, Artificial Sequence
  synthetically generated oligonucleotide
gacccttggaattcnnnnnnnnrggctagctacaacgannnnnnnnctaattaagcttcgg

SEQ ID NO: 53 - DNA, 21 bp, Artificial Sequence
  primer
ttatggtttctgagaacagat

SEQ ID NO: 54 - DNA, 21 bp, Artificial Sequence
  primer
accctggaagtggtgcagcca

SEQ ID NO: 55 - DNA, 21 bp, Artificial Sequence
  primer
atgcaccaaaagagaactgca

SEQ ID NO: 56 - DNA, 21 bp, Artificial Sequence
  primer
atggaaagtaaagatgcctcc

SEQ ID NO: 57 - DNA, 21 bp, Artificial Sequence
  primer
atggagccagtagatcctcgt

SEQ ID NO: 58 - DNA, 21 bp, Artificial Sequence
  primer
atgaactcactggtttcttgg

SEQ ID NO: 59 - DNA, 21 bp, Artificial Sequence
  primer
catgccccgcggcgcgccatt

SEQ ID NO: 60 - DNA, 21 bp, Artificial Sequence
  primer
cagttctgcgcacgcgcgcgg

SEQ ID NO: 61 - DNA, 21 bp, Artificial Sequence
  primer
cactagtaacggccgccagtg

SEQ ID NO: 62 - DNA, 21 bp, Artificial Sequence
  primer
cccttccttcgggcctgtcgg

SEQ ID NO: 63 - DNA, 21 bp, Artificial Sequence
  primer
tcactgccccgcacctgcgcc

SEQ ID NO: 64 - DNA, 21 bp, Artificial Sequence
  primer
caatctttccaggttttattc

SEQ ID NO: 65 - DNA, 38 bp, Artificial Sequence
  primer
gccaagctatttaggtgacactataggttccaggatcc

SEQ ID NO: 66 - DNA, 15 bp, Artificial Sequence
  primer
gccaagctatttagg

SEQ ID NO: 67 - DNA, 19 bp, Artificial Sequence
  primer
gtcaaaagccactgtgtcc

SEQ ID NO: 68 - DNA, 20 bp, Artificial Sequence
  primer
acaaccgaagcgtagggtca

SEQ ID NO: 69 - DNA, 31 bp, Artificial Sequence
  probe
acgaaattaggcagaaaacgactgatgagtc

SEQ ID NO: 70 - DNA, 24 bp, Artificial Sequence
  primer
gttccaaagctggatatccgctgc

SEQ ID NO: 71 - DNA, 21 bp, Artificial Sequence
  primer
cggtaccgtcagctcgacctc

SEQ ID NO: 72 - DNA, 20 bp, Artificial Sequence
  primer
gcggccgcataggaacgcgt

SEQ ID NO: 73 - DNA, 21 bp, Artificial Sequence
  primer
cacggtcagcagaatgtcatc

SEQ ID NO: 74 - DNA, 18 bp, Artificial Sequence
  primer
gatccagagatctgatga

SEQ ID NO: 75 - DNA, 27 bp, Artificial Sequence
  primer
aattctggagttactttcgtcctcacg

SEQ ID NO: 76 - RNA, 17 nt, Artificial Sequence
  target sequence
cccagaaaguuaccaca

SEQ ID NO: 77 - RNA, 17 nt, Artificial Sequence
  target sequence
uuaccacaguuaugcac

SEQ ID NO: 78 - RNA, 17 nt, Artificial Sequence
  target sequence
gacgugagguauaugac

SEQ ID NO: 79 - RNA, 17 nt, Artificial Sequence
  target sequence
cauaguauauagagaug

SEQ ID NO: 80 - RNA, 17 nt, Artificial Sequence
  target sequence
auuagugaguauagaca

SEQ ID NO: 81 - RNA, 17 nt, Artificial Sequence
  target sequence
uauaguuuguauggaac

SEQ ID NO: 82 - RNA, 17 nt, Artificial Sequence
  target sequence
uauaaggggucggugga

SEQ ID NO: 83 - RNA, 17 nt, Artificial Sequence
  target sequence
guggaccggucgaugua

SEQ ID NO: 84 - RNA, 17 nt, Artificial Sequence
  target sequence
accuaaagguugugugg

SEQ ID NO: 85 - RNA, 17 nt, Artificial Sequence
  target sequence
uagacacuuuaauuaug

SEQ ID NO: 86 - RNA, 17 nt, Artificial Sequence
  target sequence
guggaagggucguugcu

SEQ ID NO: 87 - RNA, 17 nt, Artificial Sequence
  target sequence
gaagacuuguuacccua

SEQ ID NO: 88 - RNA, 17 nt, Artificial Sequence
  target sequence
ccuguaggguuacauug

SEQ ID NO: 89 - RNA, 17 nt, Artificial Sequence
  target sequence
gaagacagcucagaaga

SEQ ID NO: 90 - RNA, 17 nt, Artificial Sequence
  target sequence
auuaccaaauacugacc

SEQ ID NO: 91 - RNA, 17 nt, Artificial Sequence
  target sequence
caauugcuauuguaaaa

SEQ ID NO: 92 - RNA, 17 nt, Artificial Sequence
  target sequence
ucagaacagucagacuc

SEQ ID NO: 93 - RNA, 17 nt, Artificial Sequence
  target sequence
ucaagcuucucuaucaa

SEQ ID NO: 94 - RNA, 17 nt, Artificial Sequence
  target sequence
gcugagccgucggggga

SEQ ID NO: 95 - RNA, 17 nt, Artificial Sequence
  target sequence
aguguuucuucugcuuc

SEQ ID NO: 96 - RNA, 17 nt, Artificial Sequence
  target sequence
aagaagcaguuugaaga

SEQ ID NO: 97 - RNA, 17 nt, Artificial Sequence
  target sequence
agccaugucucgaagau

SEQ ID NO: 98 - RNA, 17 nt, Artificial Sequence
  target sequence
uccagaaggucgcuuau

SEQ ID NO: 99 - DNA, 767 bp, Human papillomavirus
  misc_feature (1)...(767): n = A,T,C or G
gggatggaaagtaaagatgcctccacgtctgcaacatccatagaccagttgtgcaagacgtttaatctttctttgcacactctgcaaattcagtgcgtgttttgcaggaatgcactgaccaccgcagagatatatgcatatgcctataagaacctaaaggttgtgtggcgagacaactttccctttgcagcgtgtgcctgttgcttagaactgcaagggaaaattaaccaatatagacactttaattatgctgcatatgcacctacagtagaagaagaaactaatgaagatattttaaaagtgttaattcgttgttacctgtgtcacaagccgttgtgtgaaatagaaaaactaaagcacatattggaaaaggcacgcttcataaaactaaataaccagtggaagggtcgttgcttacactgctggacaacatgcatggaagacttgttaccctaaaggatatagtactagacctgcagcctcctgaccctgtagggttacattgctatgagcaattagaagacagctcagaagatgaggtggacaaggtggacaaacaagactcacagcctttaacacaacattaccaaatactgacctgttgctgtggatgtgacagcaacgtccgactggttgtggagtgcacagacggagacatnagacaactacaagaccttttgctgggcacactaaatattgtgtgtcccatctgcgcaccaaaaccataacaagggcgaattccagcacactggcggccgttactagtg

SEQ ID NO: 100 - DNA, 23 bp, Artificial Sequence
  primer
atggaaagtaaagatgcctccac

SEQ ID NO: 101 - DNA, 25 bp, Artificial Sequence
  primer
cccatctgcgcaccaaaaccataac

SEQ ID NO: 102 - DNA, 22 bp, Artificial Sequence
  primer
ctaaaggttgtgtggcgagaca

SEQ ID NO: 103 - DNA, 25 bp, Artificial Sequence
  primer
gcttagaactgcaagggaaaattaa

SEQ ID NO: 104 - DNA, 25 bp, Artificial Sequence
  probe
ctttccctttgcagcgtgtgcctgt